(19) United States
(12) Patent Application Publication
(10) Pub. No.: US 2014/0286977 A1
(43) Pub. Date: Sep. 25, 2014

(54) Protein F - A Novel Haemophilus Influenzae Adhesin with Laminin and Vitronectin binding Properties

(71) Applicants: Kristian Riesbeck, Malmo (SE); Yu-Ching Su, Malmo (SE)

(72) Inventors: Kristian Riesbeck, Malmo (SE); Yu-Ching Su, Malmo (SE)

(73) Assignee: Riesbeck Healthcare Sweden AB, Malmo (SE)

(21) Appl. No.: 14/116,968

(22) PCT Filed: May 11, 2012
(86) PCT No.: PCT/SE2012/050503

§ 371 Date: Apr. 29, 2014

Related U.S. Application Data
(60) Provisional application No. 61/484,697, filed on May 11, 2011.

(30) Foreign Application Priority Data
May 11, 2011 (SE) 1150418-0

(51) Int. Cl. A61K 39/145; A61K 39/09; A61K 39/02; C12P 21/02

(52) U.S. Cl. CPC A61K 39/145; C12P 21/02; A61K 39/092; A61K 39/0208
USPC 424/186.1; 435/69.3

(57) ABSTRACT

A vaccine composition comprising a protein, which can be detected in Haemophilus influenzae, having an amino acid sequence as described in SEQ ID NO: 1, or a fragment thereof, is described. The fragment comprises an amino acid sequence having at least 15 contiguous amino acids from the amino acid sequence of SEQ ID NO: 1, and the fragment (if necessary when coupled to a carrier) is capable of raising an immune response which recognises the polypeptide of SEQ ID NO: 1.

### FIELD OF THE INVENTION

The present invention relates to a laminin and vitronectin binding protein with adhesive properties (protein F; pF), a virulence factor, which can be found in Haemophilus influenzae. The present invention further relates to vaccine compositions comprising protein F.

### BACKGROUND OF THE INVENTION

Both Haemophilus influenzae type b (Hib) and nontypeable H. influenzae (NTHi) cause a variety of diseases in children and in adults. Hib causes bacterial meningitis and other invasive infections in children under the age of 4 years, whereas NTHi has been isolated from cases of otitis media, sinusitis, epiglottitis, tracheobronchitis, and pneumonia and may cause neonatal sepsis. There is currently no commercially available vaccine against NTHi, but a number of vaccines are in use against Hib. These vaccines consist of the Hib capsular polysaccharide, polyribosyl ribitol phosphate, conjugated to various protein carriers (meningococcal outer membrane complex, tetanus toxoid, nontoxic mutant diphtheria toxin, or diphtheria toxoid) to overcome the weak immune response to capsular polysaccharide in children younger than 18 months of age. H. influenzae outer membrane proteins (OMPs) are also considered to be carriers of polyribosyl ribitol phosphate since they are shown to be targets of host antibodies following Hib and NTHi infections. Antibodies to OMPs P1, P2, P4, P5, and P6 and a 98-kDa protein have been tested in in vivo protection and in vitro bactericidal assays against H. influenzae infections, with antibodies to P1, P4, and P6 showing biological activity against both homologous and heterologous H. influenzae strains. The lack of heterologous protection from antibodies to other OMPs is partly due to the antigenic diversity of these proteins among different H. influenzae strains. An ideal antigen must therefore be both exposed on the bacterial surface and antigenically well conserved. For example, 42-kDa membrane protein (protein D) that is widely distributed and antigenically conserved among both Hib and NTHi strains has been isolated, cloned, sequenced, and shown to be a pathogenicity factor and a valid vaccine candidate (Janson et al., 1991, Prymula et al., 2006).

An initial step in NTHi pathogenesis is adherence to the mucosa, basement membrane and to the extracellular matrix (ECM). Two main classes of macromolecules constitute the ECM of mammals, the fibrous proteins that have both structural and adhesive functions (e.g. laminin, collagens, and elastin) and the glycosaminoglycans that are linked to proteins in the form of proteoglycans [Heino et al., 2009]. The ECM stabilizes the physical structure of tissue, is involved in regulating eukaryotic cell adhesion, differentiation, migration, proliferation, shape and structure. Bacterial interaction with the ECM plays an important role in colonization of host tissues, and the ECM is not exposed to pathogens under normal circumstances. However, after tissue damage due to a mechanical or chemical injury or a bacterial/viral infection through the activity of toxins and lytic enzymes, the pathogen may gain access to the ECM.

Laminins are a family of heterotrimeric, large cruciformed shaped glycoproteins of approximately 400-900 kDa consiting of a α, β, and γ chain [Nguyen 2006]. There are different α, β, and γ chains and these combine into different laminin isoforms. The major role of laminin for the epithelium is to anchor cells to the basal membrane. Several pathogens bind laminin, for example, Mycobacterium tuberculosis [Kinhikar et al., 2006] and Moraxella catarrhalis [Tan et al. 2006].

Vitronectin is another important component of the ECM, and is synthesized in the liver and secreted into plasma [for a review see Singh et al., 2010b]. Most of the circulating vitronectin in blood is a monomer (65 and 75 kDa), whereas the extravascular cell-bound vitronectin is a multimer. Vitronectin is found at a high concentration in plasma (200-700 μg/ml), and is also present in different human tissues. Particularly high amounts are observed in liver, tonsil, duodenum, heart, skeletal muscle, and lung tissues.

Vitronectin plays a crucial role in many biological processes including cell migration, adhesion and angiogenesis [Preissner et al., 1998]. The interaction of vitronectin with the urokinase plasminogen activator-urokinase plasminogen activator receptor (uPA-uPAR) complex and integrin receptors is a part of the plasminogen activation system involved in old tissue degradation (pericellular proteolysis), reorganization and wound healing. Hence the uPAR-vitronectin interaction is a key determinant in homeostatic processes [Smith et al., 2010].

In addition to being a component of the ECM, vitronectin is involved in regulation of the terminal pathway of complement activation to limit the self-reactivity of the innate immune response. Formation of the membrane attack complex (MAC) is under control of the two inhibitors membrane protein CD59 and vitronectin. Vitronectin binds the C5b-7 complex at its membrane-binding site and thereby inhibits the insertion of the complex into the cell membrane. Therefore, formation of cytolytic MAC is inhibited and lysis of the cell prevented [Preissner, 1991]. In the presence of vitronectin, C5b-7 complex is still able to bind C8 and C9 to form C5b-8 and C5b-9 complexes but the latter is non-lytic. Furthermore, vitronectin blocks pore-forming polymerization of C9 by binding C5b-9 [Preissner et al., 1985]. In addition to utilize vitronectin as a bridge for attachment to epithelial cells, several pathogens including NTHi use vitronectin as an efficient complement regulator for inhibition of the MAC in order to increase survival in human serum [Singh et al., 2010b]. Several bacterial outer membrane proteins, amongst others, M. catarrhalis ubiquitous surface protein (UspA2) [Singh et al., 2010] and H. influenzae protein E [Hallström et al. 2009] have recently been shown to interact with vitronectin.

In WO2007/084053 a surface exposed protein named protein E, which protein can be detected in Haemophilus influenzae, is described. Protein E is an adhesin and also binds vitronectin [Ronander 2009, Hallström 2009].

### SUMMARY OF THE INVENTION

In view of the fact that H. influenzae has been found to be such a leading cause of infections in the upper and lower airways, there is a current need to develop vaccines that can be used against H. influenzae.

The aim of the present invention has therefore been to further study the way which way H. influenzae interacts with cells in the body and interacts with the immune system, to be able to provide a new type of effective vaccine against H. influenzae.

According to one aspect, the present invention provides a surface exposed protein, which can be detected in Haemophilus influenzae, having an amino acid sequence according to Sequence ID No. 1, or a fragment thereof, wherein the fragment comprises an amino acid sequence having at least 15 contiguous amino acids from the amino acid sequence of SEQ ID NO: 1 which fragment (if necessary when coupled to a carrier) is capable of raising an immune response which recognises the polypeptide of SEQ ID NO: 1.

According to another aspect the present invention provides a vaccine composition comprising an immunogenic fragment of the surface exposed protein according to claim 1, which fragment can be detected in Haemophilus influenzae.

According to a further aspect the present invention provides a vaccine composition comprising an immunogenic protein of the based on the protein mentioned above, wherein one or more of the amino acids in position 1-11 or 1-22 of SEQ ID No. 1 have been deleted or replaced by one or more amino acids. In one embodiment one or more of the amino acids in position 1-11 or 1-22 of SEQ ID No. 1 have been replaced by a sequence of 0-11 or 0-22 optional amino acids.

According to a further aspect the present invention provides a vaccine composition comprising peptide having an amino acid sequence according to Sequence ID NO: 2, or a fragment, wherein the fragment comprises an amino acid sequence having at least 15 contiguous amino acids from the amino acid sequence of SEQ ID NO:2 which fragment (if necessary when coupled to a carrier) is capable of raising an immune response which recognises the polypeptide of SEQ ID NO:2.

According to still a further aspect the present invention provides a vaccine composition comprising a peptide having an amino acid sequence according to Sequence ID NO: 3, or a fragment, wherein the fragment comprises an amino acid sequence having at least 15 contiguous amino acids from the amino acid sequence of SEQ ID NO:3 which fragment (if necessary when coupled to a carrier) is capable of raising an immune response which recognises the polypeptide of SEQ ID NO:3.

According to yet a further aspect the present invention provides a vaccine composition comprising a peptide having an amino acid sequence according to Sequence ID NO: 4, or a fragment, wherein the fragment comprises an amino acid sequence having at least 15 contiguous amino acids from the amino acid sequence of SEQ ID NO:4 which fragment (if necessary when coupled to a carrier) is capable of raising an immune response which recognises the polypeptide of SEQ ID NO:4.

According to a further aspect the present invention provides a vaccine composition comprising a peptide having an amino acid sequence according to Sequence ID NO: 5, or a fragment, wherein the fragment comprises an amino acid sequence having at least 15 contiguous amino acids from the amino acid sequence of SEQ ID NO:5 which fragment (if necessary when coupled to a carrier) is capable of raising an immune response which recognises the polypeptide of SEQ ID NO:5.

According to another aspect the present invention provides a vaccine composition comprising a peptide having an amino acid sequence according to Sequence ID NO: 6, or a fragment, wherein the fragment comprises an amino acid sequence having at least 15 contiguous amino acids from the amino acid sequence of SEQ ID NO:6 which fragment (if necessary when coupled to a carrier) is capable of raising an immune response which recognises the polypeptide of SEQ ID NO:6.

According to still a further aspect the present invention provides a vaccine composition comprising a peptide having an amino acid sequence according to Sequence ID NO: 7, or a fragment, wherein the fragment comprises an amino acid sequence having at least 15 contiguous amino acids from the amino acid sequence of SEQ ID NO:7 which fragment (if necessary when coupled to a carrier) is capable of raising an immune response which recognises the polypeptide of SEQ ID NO:7.

According to another aspect the present invention provides a vaccine composition comprising a peptide having an amino acid sequence according to Sequence ID NO: 8, or a fragment, wherein the fragment comprises an amino acid sequence having at least 15 contiguous amino acids from the amino acid sequence of SEQ ID NO:8 which fragment (if necessary when coupled to a carrier) is capable of raising an immune response which recognises the polypeptide of SEQ ID NO:8.

According to yet a further aspect the present invention provides a vaccine composition comprising a peptide having an amino acid sequence according to Sequence ID NO: 9, or a fragment, wherein the fragment comprises an amino acid sequence having at least 15 contiguous amino acids from the amino acid sequence of SEQ ID NO:9 which fragment (if necessary when coupled to a carrier) is capable of raising an immune response which recognises the polypeptide of SEQ ID NO:9.

According to a further aspect the present invention provides a vaccine composition comprising a peptide having an amino acid sequence according to Sequence ID NO: 10, or a fragment, wherein the fragment comprises an amino acid sequence having at least 15 contiguous amino acids from the amino acid sequence of SEQ ID NO:10 which fragment (if necessary when coupled to a carrier) is capable of raising an immune response which recognises the polypeptide of SEQ ID NO:10.

According to another aspect the present invention provides a vaccine composition comprising a peptide having an amino acid sequence according to Sequence ID NO: 11, or a fragment, wherein the fragment comprises an amino acid sequence having at least 15 contiguous amino acids from the amino acid sequence of SEQ ID NO:11 which fragment (if necessary when coupled to a carrier) is capable of raising an immune response which recognises the polypeptide of SEQ ID NO:11.

According to still a further aspect the present invention provides a vaccine composition comprising a peptide having an amino acid sequence according to Sequence ID NO: 12, or a fragment, wherein the fragment comprises an amino acid sequence having at least 15 contiguous amino acids from the amino acid sequence of SEQ ID NO:12 which fragment (if necessary when coupled to a carrier) is capable of raising an immune response which recognises the polypeptide of SEQ ID NO:12.

According to a further aspect the present invention provides a vaccine composition comprising a peptide having an amino acid sequence according to Sequence ID NO: 13, or a fragment, wherein the fragment comprises an amino acid sequence having at least 15 contiguous amino acids from the amino acid sequence of SEQ ID NO:13 which fragment (if necessary when coupled to a carrier) is capable of raising an immune response which recognises the polypeptide of SEQ ID NO:13.

According to another aspect the present invention provides a vaccine composition comprising a peptide having an amino acid sequence according to Sequence ID NO: 14, or a fragment, wherein the fragment comprises an amino acid sequence having at least 15 contiguous amino acids from the amino acid sequence of SEQ ID NO:14 which fragment (if necessary when coupled to a carrier) is capable of raising an immune response which recognises the polypeptide of SEQ ID NO:14.

According to one aspect the present invention provides a vaccine composition comprising at least one di-, tri- or multimer of a protein or fragment according to above.

According to a further aspect the present invention provides a vaccine composition according to above, further comprising one or more pharmaceutically acceptable adjuvants, vehicles, excipients, binders, carriers, preservatives, buffering agents, emulsifying agents, wetting agents, or transfection facilitating compounds.

In another aspect the present invention provides a vaccine composition according to above, comprising at least one further vaccine.

According to a further aspect the present invention provides a vaccine composition according to above, comprising an immunogenic portion of another molecule.

According to a further aspect the present invention provides a vaccine composition as described above, wherein the immunogenic portion of another molecule is chosen from the group comprising Protein D, pilA or protein E of H. influenzae, MID of Moraxella catarrhalis, UspA1 or UspA2 of Moraxella catarrhalis, and outer membrane protein of any respiratory tract pathogen found in humans or animals.

According to a further aspect the present invention provides a vaccine composition comprising a nucleic acid sequence encoding a protein or fragment as described above, as well as homologues, polymorphisms, degenerates and splice variants thereof.

According to another aspect the present invention provides a vaccine composition comprising a recombinant nucleic acid sequence comprising a nucleic acid sequence according to above, which is fused to at least another gene.

According to yet a further aspect the present invention provides a vaccine composition comprising a plasmid or phage comprising a nucleic acid sequence as described above.

According to yet another aspect the present invention provides a vaccine composition comprising a non human host comprising at least one plasmid according to above and capable of producing a protein or fragment as mentioned above, which host is chosen among bacteria, yeast and plants.

According to a further aspect the present invention provides a vaccine composition comprising a host as described above, which is E. coli.

According to a further aspect the present invention provides a vaccine composition comprising a fusion protein or polypeptide, in which a protein or fragment according to above is combined with at least another protein by the use of a recombinant nucleic acid sequence as mentioned above.

According to another aspect the present invention provides a vaccine composition comprising a fusion protein according to above, which is a di-, tri or multimer of a protein or fragment as mentioned above.

According to one aspect the present invention provides a vaccine composition comprising a fusion product, in which a protein or fragment or peptide according to above is covalently, or by any other means, bound to a protein, carbohydrate or matrix.

According to another aspect the present invention provides a vaccine composition comprising an isolated polypeptide comprising an amino acid sequence which has at least 85% identity to the amino acid sequence of SEQ ID NO:1 over the entire length of SEQ ID NO:1.

According to a further aspect the present invention provides a vaccine composition comprising an isolated polypeptide as claimed in claim 31 in which the amino acid sequence has at least 95% identity to the amino acid sequence of SEQ ID NO:1.

According to yet a further aspect the present invention provides a vaccine composition as described above comprising the amino acid sequence of SEQ ID NO:1.

According to a further aspect the present invention provides a vaccine composition comprising an isolated polypeptide of SEQ ID NO:1.

According to another aspect the present invention provides a vaccine composition as described above, wherein the polypeptide lacks a signal peptide (amino acids 1-22) of SEQ ID NO: 1.

In one embodiment the present invention provides a vaccine composition comprising an immunogenic fragment comprising an amino acid sequence having at least 15 contiguous amino acids from the amino acid sequence of SEQ ID NO:1 or from the polypeptide as described above, which fragment (if necessary when coupled to a carrier) is capable of raising an immune response which recognises the polypeptide of SEQ ID NO:1 (or the polypeptide as described above, respectively), or is capable of binding vitronectin and laminin.

According to a further aspect the present invention provides a vaccine composition comprising a polypeptide or an immunogenic fragment as described above, wherein said polypeptide or said immunogenic fragment is part of a larger fusion protein.

According to another aspect the present invention provides a vaccine composition comprising an isolated polynucleotide encoding a polypeptide or an immunogenic fragment as described above.

According to a further aspect the present invention provides a vaccine composition comprising an isolated polynucleotide comprising a nucleotide sequence encoding a polypeptide that has at least 85% identity to the amino acid sequence of SEQ ID NO:1 over the entire length of SEQ ID NO:1; or a nucleotide sequence complementary to said isolated polynucleotide.

According to another aspect the present invention provides a vaccine composition comprising an isolated polynucleotide comprising a nucleotide sequence that has at least 85% identity to a nucleotide sequence encoding a polypeptide of SEQ ID NO:1 over the entire coding region; or a nucleotide sequence complementary to said isolated polynucleotide.

According to yet a further aspect the present invention provides a vaccine composition comprising an isolated polynucleotide which comprises a nucleotide sequence which has at least 85% identity to that of SEQ ID NO: 15 over the entire length of SEQ ID NO: 15; or a nucleotide sequence complementary to said isolated polynucleotide.

According to another aspect the present invention provides a vaccine composition as described above in which the identity of the isolated polynucleotide is at least 95% to SEQ ID NO:15.

In one embodiment the present invention provides a vaccine composition comprising an isolated polynucleotide comprising a nucleotide sequence encoding the polypeptide of SEQ ID NO:1, or an immunogenic fragment as described above.

According to a further aspect the present invention provides a vaccine composition comprising an isolated polynucleotide comprising the polynucleotide of SEQ ID NO:15.

According to yet a further aspect the present invention provides a vaccine composition comprising an isolated polynucleotide comprising a nucleotide sequence encoding the polypeptide of SEQ ID NO:1, obtainable by screening an appropriate library under stringent hybridization conditions with a labeled probe having the sequence of SEQ ID NO: 15 or a fragment thereof.

In a further aspect the present invention provides a vaccine composition comprising an expression vector or a recombinant live microorganism comprising an isolated polynucleotide according to above.

In yet a further aspect the present invention provides a vaccine composition comprising a recombinant live microorganism comprising an expression vector according to above.

According to a further aspect the present invention provides a vaccine composition comprising a host cell comprising the expression vector as described above.

According to another aspect the present invention provides a vaccine composition comprising a membrane of the host cell according to above expressing an isolated polypeptide comprising an amino acid sequence that has at least 85% identity to the amino acid sequence of SEQ ID NO:1.

According to yet a further aspect the present invention provides a process for producing a vaccine composition according to above comprising culturing a host cell as described above under conditions sufficient for the production of said polypeptide or said immunogenic fragment and recovering the polypeptide from the culture medium.

In one embodiment the present invention provides a process for producing a vaccine composition according to above, comprising transforming a host cell with an expression vector comprising at least one of said polynucleotides and culturing said host cell under conditions sufficient for expression of any one of said polynucleotides.

In another embodiment the present invention provides a vaccine composition comprising an effective amount of the polypeptide or the immunogenic fragment of above and a pharmaceutically acceptable excipient.

In yet another embodiment the present invention provides a vaccine composition comprising an effective amount of the polynucleotide as described above and a pharmaceutically acceptable excipient.

According to one aspect the present invention provides a vaccine composition according to above, wherein said composition comprises at least one other Haemophilus influenzae antigen.

According to a further aspect the present invention provides a vaccine composition according to above, formulated with pneumolysin from Streptococcus pneumoniae.

According to yet a further aspect the present invention provides a vaccine composition as mentioned above, formulated with Omp106 from Moraxella catarrhalis.

According to a further aspect the present invention provides a vaccine composition according to above, formulated with UspA1 and/or UspA2 from Moraxella catarrhalis.

In one embodiment the present invention provides a vaccine composition according to above, formulated with Hly 3 from Moraxella catarrhalis.

In another embodiment the present invention provides a vaccine composition as mentioned above, formulated with OmpCD from Moraxella catarrhalis.

According to a further aspect the present invention provides a vaccine composition according to above, formulated with D15 from Moraxella catarrhalis.

According to another aspect the present invention provides a vaccine composition according to above, formulated with Omp 26 from Haemophilus influenzae.

According to yet another aspect the present invention provides a vaccine composition as mentioned above, formulated with P6 from Haemophilus influenzae.

According to a further aspect the present invention provides a vaccine composition according to above, formulated with Protein D or E from Haemophilus influenzae.

According to a further aspect the present invention provides a vaccine composition according to above, formulated with NIpC2 from Haemophilus influenzae.

According to yet a further aspect the present invention provides a vaccine composition as mentioned above, formulated with Slp or pilA from Haemophilus influenzae.

In one embodiment the present invention provides use of a vaccine according to above in the manufacture of a medicament for the prophylaxis or treatment of an infection.

In a further embodiment the present invention provides use according to above, wherein the infection is caused by Haemophilus influenzae.

According to a further aspect the present invention provides use according to above, wherein the Haemophilus influenzae is encapsulated or non-typable.

According to yet a further aspect the present invention provides use as mentioned above for the prophylaxis or treatment of otitis media, sinusitis or lower respiratory tract infections in children and adults suffering from e.g. chronic obstructive pulmonary disease (COPD).

According to another aspect the present invention provides a medicament comprising at least one protein, fragment or peptide according to above and one or more pharmaceutically acceptable adjuvants, vehicles, excipients, binders, carriers, preservatives, buffering agents, emulsifying agents, wetting agents, or transfection facilitating compounds.

According to yet another aspect the present invention provides a method of isolation of a protein, fragment or peptide according to above, said method comprising the steps:

a) growing Haemophilus influenzae or E. coli comprising the DNA coding for said protein, fragment or peptide, harvesting the bacteria and isolating outer membranes, outer membrane vesicles or inclusion bodies;

b) solubilizing the inclusion bodies with a strong solvatising agent;

c) adding a renaturating agent; and

d) dialyzing the resulting suspension against a buffer with a pH of from 8 to 10.

According to a further aspect the present invention provides a method according to above, wherein the solvalising agent is guanidium hydrochloride.

According to yet a further aspect the present invention provides a method as mentioned above, wherein the renaturating agent is arginin.

According to another aspect the present invention provides a method of making a vaccine as mentioned above, wherein the protein, fragment or peptide is formulated with an excipient.

According to a further aspect the present invention provides a method of preventing or treating an infection in an individual comprising administering a pharmaceutically effective amount of a vaccine composition according to above.

In one embodiment said infection is caused by Haemophilus influenzae, both encapsulated or non-typable, and in yet another embodiment the infection is chosen from the group consisting of otitis media, sinusitis or lower respiratory tract infections.

In one aspect the present invention relates to a nucleic acid sequence encoding a protein, fragment or peptide as described above, as well as homologues, polymorphisms, degenerates and splice variants thereof. In one embodiment, said nucleic acid sequence is fused to at least another gene.

In another aspect the present invention relates to a plasmid or phage comprising a nucleic acid sequence as described above.

In yet another embodiment the present invention relates to a non human host comprising at least one plasmid as described above and capable of producing a protein, fragment or peptide as discussed above, as well as homologues, polymorphisms, degenerates and splice variants thereof, which host is chosen among bacteria, yeast and plants. In one embodiment, said host is E. coli.

In still another aspect, the present invention provides a fusion protein or polypeptide, in which a protein, fragment or peptide as described above is combined with at least another protein by the use of a recombinant nucleic acid sequence as discussed above. In one embodiment, said fusion protein is a di-, tri or multimer of a protein, fragment or peptide as discussed above.

In one aspect, the present invention relates to a fusion product, in which a protein, fragment or peptide as described above is covalently, or by any other means, bound to a protein, carbohydrate or matrix.

The present invention relates to Protein F, in particular Protein F polypeptides and Protein F polynucleotides, recombinant materials and methods for their production. In another aspect, the invention relates to methods for using such polypeptides and polynucleotides, including prevention and treatment of microbial diseases, amongst others. In a further aspect, the invention relates to diagnostic assays for detecting diseases associated with microbial infections and conditions associated with such infections, such as assays for detecting expression or activity of Protein F polynucleotides or polypeptides.

Various changes and modifications within the spirit and scope of the disclosed invention will become readily apparent to those skilled in the art from reading the following descriptions and from reading the other parts of the present disclosure.

### DESCRIPTION OF THE FIGURES

FIG. 1. A 29 kDa vitronectin-binding H. influenzae protein is detected both in 1- and 2-dimensional gel electrophoresis (2D-SDS-PAGE) using vitronectin as bate. Outer membrane vesicles (OMV) were prepared from the clinical isolate NTHi 3655 in the absence (−) or presence (+) of CO₂. An SDS-PAGE was run and blotted to a nylon filter (left panels). OMV from the culture incubated in the presence of CO₂ were also subjected to a 2D-SDS-PAGE (right panels). For both the 1- and 2-dimensional gels, two corresponding gels were run in parallel. One gel was blotted to a nylon filter (A), and one gel was stained with Coomassie blue (B). Human plasma vitronectin was added and the filters were subsequently probed with a goat anti-vitronectin pAb followed by detection using a HRP-conjugated donkey anti-sheep pAb (A: i). In panel A:ii, a filter is shown that were incubated with the primary and secondary pAb only (without vitronectin) in order to exclude background binding of the secondary antibodies. The spot corresponding to a putative vitronectin-binding protein (B) were cut out from the Coomassie stained gel and sequenced by MALDI-ToF. pF is shown with arrows in the panels A: i and B.

FIG. 2. DNA sequence and translated open reading frame including the signal peptide for protein F (SEQ ID No: 1) in NTHi 3655. In (A) the DNA sequence together with the translated protein sequence is shown, and in (B) a cartoon on pF indicating different predicted regions are outlined.

FIG. 3. Protein F is very homologous in a set of different H. influenzae strains. Different pF amino acid sequences corresponding to HI 0362 found in GeneBank were compared by Clustal analysis.

FIG. 4. Recombinant pF 12-293 produced in E. coli. H. influenzae DNA was used as a template and the sequence corresponding to the open reading frame of pF devoid of the N-terminal part (amino acids pF 1-11) of the predicted signal peptide was amplified by PCR and cloned into pET26 followed by transformation of E. coli and subsequent induction, expression and purification by affinity chromatography using a Ni-resin. In (A), a Coomassie stained gel is shown, and in (B) a Western blot with pF detected using a rabbit anti-pF serum and HRP-conjugated swine anti-rabbit pAb. Outer membrane vesicles (OMV) from NTHi 3655 and M. catarrhalis Bc5 were included as a positive and negative control, respectively. The M. catarrhalis OMV proved the specificity of the rabbit antiserum.

FIG. 5. Protein F can be found in the outer membrane of non-typable H. influenzae. (A) Coomassie stained gel with clinical NTHi isolates are indicated. (B) Western blot showing the location of pF. Outer membrane proteins (OMPs) from eight different clinical NTHi isolates were subjected to SDS-PAGE and analysed for pF expression using the specific rabbit anti-pF antiserum.

FIG. 6. A pF-deficient NTHi 3655 mutant does not express pF at the surface. (A) Flow cytometry profile of NTHi 3655 wild type (WT) showing background values with the goat anti-rabbit pAb-FITC in the absence of the primary anti-pF rabbit pAb. (B) Protein F expression on NTHi 3655 as judged by specific anti-pF pAb. (C) Fluorescence of the NTHi 3655 Δpf mutant in the absence of the anti-pF rabbit pAb. (D) The pF-deficient NTHi 3655 Δpf mutant did not express pF at the surface. An NTHi 3655 Δpf mutant containing a cat gene was manufactured as described in Material and Methods. The resulting mutant and wild type bacterium were grown in broth overnight and washed. Thereafter, primary and detection pAb were added as indicated followed by incubation on ice, washes and finally analysis in a flow cytometer. The anti-pF antiserum was raised in rabbits. After 3 immunizations with recombinant pF 12-293 and adjuvants, the resulting rabbit antiserum was immunopurified against pF 12-293. One typical experiment out of three with similar results is shown.

FIG. 7. Recombinant pF (amino acids 12-293) binds vitronectin and a pF-expressing NTHi 3655 binds significantly more vitronectin as compared to a pF-deficient mutant NTHi 3655 Δpf. (A) The vitronectin-binding to pF 12-293 was slightly better as compared to recombinant H. influenzae protein E, a recently published adhesin [Ronander et al., 2009]. (B) An H. influenzae mutant devoid of pF has a significantly reduced binding to vitronectin as compared to the wild type bacterium. In (A), recombinant proteins were coated in microtiterplates and analysed for vitronectin-binding using human vitronectin and a rabbit anti-vitronectin pAb. The highly vitronectin-binding M. catarrhalis protein UspA2 30-539 [Singh et al., 2010a] was included as a positive control. MID 962-1200 indicates a negative control that is a recombinantly expressed protein derived from M. catarrhalis strain “Bc5”. Bovine serum albumin (BSA) was another negative control. In (B), NTHi 3655 and the pF-mutant NTHi 3655 Δpf were incubated with [¹²⁵I]-labelled vitronectin. After 30 mins, bacteria were washed and subjected to measurement in a γ-scintillation counter. The data represent 3 independent experiments each in panels A and B.

FIG. 8. Protein F 12-293 binds the ECM protein laminin. Recombinant pF or pE were attached to a plastic surface in a microtiter plate. Mouse sarcoma laminin was added followed by detection using a rabbit anti-laminin pAb and a HRP-conjugated goat anti-rabbit pAb as a secondary layer. BSA was included as a negative control. Mean values of 3 independent experiments are shown.

FIG. 9. Recombinant pF12-293 binds to epithelial cells. The type II alveolar epithelial cell line A549 were grown to confluency in 24-well plates, washed and fixed with formaldehyde. Thereafter, recombinant pF12-293 at increasing concentrations was incubated with the cells followed by extensive washing steps. Protein F was detected with the anti-pF rabbit antiserum and HRP-conjugated goat anti-rabbit pAb as a secondary layer. A typical experiment out of three independent ones are shown.

FIG. 10. A pF-deficient NTHi mutant has a significantly decreased binding capacity to epithelial cells when compared with the pF-expressing wild type. A549 epithelial cells were grown to confluency in 24-well plates. Bacteria were cultured for 3 hrs in the presence of [³H]-thymidine. The NTHi 3655 wild type (WT) or pF-deficient mutant (Δpf) were added to cells followed by incubation for 2 hrs. Thereafter, cells were washed 3 times, trypsinized and measured in beta-scintillation counter. Different multiplies of infection (MOI) were used. p values were obtained by Student's t test for paired data. Mean values ±SD for three independent experiments are shown.

FIG. 11. Layout of peptides that were used for mapping of the epithelial cell binding region of pF as demonstrated in FIG. 12.

FIG. 12. Mainly pF amino acid residues 23-48 bind to epithelial cells. Results are shown for (A) H292 and (B) A549 epithelial cell lines. Cells were grown in T25 flasks to confluency and detached by trypsin-EDTA followed by washing. Thereafter, a direct binding assay (DBA) was performed. [¹²⁵I]-labelled peptides were added in molar ratios and incubated with cells at +4° C. for 30 mins. Radioactivity was measured in a γ-scintillation counter.

FIG. 13. Anti-pF44-68 pAb recognizes pF at the surface of NTHi 3655 when analysed by flow cytometry. The pF-expressing NTHi 3655 wild type (A and B) was compared to the pF-deficient NTHi 3655 Δpf mutant (C and D). (A) and (C) indicate background values in the absence of specific anti-pF44-68 pAb. Antibodies recognizing pF44-68 were immunopurified from the anti-pF 12-293 antiserum using the peptide pF44-68 attached to a CnBr-Sepharose column. pF44-68 is a predicted immunogenic region as revealed by bioinformatics (not shown). Primary and detection pAb were added as indicated followed by incubation on ice, washes and finally analysis in a flow cytometer.

### DESCRIPTION OF THE INVENTION

Before explaining the present invention in detail, it is important to understand that the invention is not limited in this application to the details of the embodiments and steps described herein. The examples mentioned are illustrative of the invention but do not limit it in any way. The invention is capable of other embodiments and of being practiced or carried out in a variety of ways. It is to be understood that the phraseology and terminology employed herein is for the purpose of description only and not of limitation.

The present application describes the cloning and expression of a novel H. influenzae outer membrane protein designated protein F (pF). The protein was discovered using human vitronectin as a bate.

To maximize the yield of recombinant pF, a truncated pF fragment consisting of amino acid residues A12 to K293 was manufactured. The N-terminal part of the predicted signal peptide including amino acids pF1-11 was thus removed and replaced with the leader peptide in addition to nine residues originating from the vector pET26(+). The truncated pF was designated pF12-293.

The present invention comprises the Haemophilus outer membrane protein pF and the pF-derived peptides pF23-48 (SEQ ID No:2), pF44-68 (SEQ ID No:3), pF64-88 (SEQ ID No:4), pF84-108 (SEQ ID No:5), pF104-128 (SEQ ID No:6), pF124-148 (SEQ ID No:7), pF144-168 (SEQ ID No:8), pF164-188 (SEQ ID No:9), pF184-209 (SEQ ID No:10), pF204-230 (SEQ ID No:11), pF225-255 (SEQ ID No:12), pF250-275 (SEQ ID No:13), pF270-293 (SEQ ID No:14), and di-, tri- or oligomers thereof. In particular, sequences of pF or the derived peptides that are surface exposed are given a higher priority.

Thus, the vaccine compositions according to the present invention comprise as immunogenic components a surface exposed protein, which can be detected in all Haemophilus influenzae, having an amino acid sequence according to SEQ ID No: 1, and/or a peptide having an amino acid sequence according to SEQ ID No: 2-14, or a fragment, homologue, functional equivalent, derivative, degenerate or hydroxylation, sulphonation or glycosylation product or other secondary processing product thereof. The vaccine compositions may also comprise a fusion protein or polypeptide, or a fusion product according to the present invention as immunogenic components. The immunogenic components are capable of eliciting an antibody or other immune response to Haemophilus influenzae, wherein the antibodies elicited inhibit the pathogenesis of Haemophilus influenzae bacterium to the cells of the subject. An “immunogenic dose” of a vaccine composition according to the invention is one that generates, after administration, a detectable humoral and/or cellular immune response in comparison to a standard immune response before administration.

The nucleic sequences used in the vaccine compositions of the present invention to generate the antigens may be inserted into any of a wide variety of expression vectors by a variety of procedures. Such procedures are deemed to be known by those skilled in the art.

Vaccine compositions are easily accomplished using well known methods and techniques, and can be administered in a variety of ways, preferably parenterally or intranasally. Formulations suitable for parenteral or intranasal administration include aqueous and non-aqueous sterile injection solutions which may contain anti-oxidants, buffers, bacteriostats and solutes that make the formulation isotonic with the bodily fluid of the subject in question; and aqueous and non-aqueous sterile suspensions which may include suspending agents or thickening agents. The active immunogenic ingredient is often mixed with excipients which are pharmaceutically acceptable, e g water, saline, dextrose, glycerol, ethanol, or the like. In addition, the vaccine composition can also contain minor amounts of auxiliary substances such as wetting or emulsifying agents, pH buffering agents, binders, carriers or preservatives.

The vaccine compositions may also include adjuvants for enhancing the immunogenicity of the composition, such as Freund's Adjuvants and other systems known in the art.

The immunogenic components of the vaccine compositions, i.e. the proteins, fragments, peptides, fusion proteins or polypeptides, or fusion products of the present invention, may be formulated into the vaccine as neutral or salt forms.

The dosage of the vaccine compositions will depend on the specific activity of the vaccine and can be readily determined by routine experimentation. The vaccine compositions are administered in such an amount as will be therapeutically effective and immunogenic, and the quantity depends on the subject.

The invention relates to Protein F polypeptides and polynucleotides as described in greater detail below. In particular, the invention relates to polypeptides and polynucleotides of Protein F of H. Influenzae. The Protein F polypeptides have a signal sequence and are exposed at the surface of the bacteria. The signal peptide is located from residue 1 to residue 22 of Protein F polypeptide.

A reference to “Protein F” herein is a reference to any of the peptides, immunogenic fragments, fusions, polypeptides or proteins of the invention discussed herein (such as SEQ ID NO: 1, with or without the signal sequence or where the amino acids in position 1-11 (the N-terminal portion of the predicted signal peptide) or 1-22 of the signal peptide may be deleted or replaced by one or more amino acids).

A “polynucleotide encoding Protein F” refers to any polynucleotide sequence encoding any of the peptides, immunogenic fragments, fusions, polypeptides or proteins of the invention discussed herein.

The term “comprising” herein alternatively may be substituted with the term “consisting of”.

It is understood that sequences recited in the Sequence Listing below as “DNA” represent an exemplification of one embodiment of the invention, since those of ordinary skill will recognize that such sequences can be usefully employed in polynucleotides in general, including ribopolynucleotides.

The sequence of the Protein F is set out in SEQ ID No: 1 (from NTHi strain 3655). The sequences of the Protein F encoded peptides, used for mapping of the epithelial cell binding region of pF, are set out in SEQ ID No: 2-14 (from NTHi strain 3655). The sequence of the Protein F polynucleotides is set out in SEQ ID NO: 15.

### Polypeptides

In one aspect of the invention there are provided polypeptides of H. influenzae (in particular non typeable H. influenzae) referred to herein as “Protein F” and “Protein F polypeptides” as well as biologically, diagnostically, prophylactically, clinically or therapeutically useful variants thereof, and compositions comprising the same.

The present invention further provides for:
(a) an isolated polypeptide which comprises an amino acid sequence which has at least 85% identity, preferably at least 90% identity, more preferably at least 95% identity, most preferably at least 97-99% or exact identity, to that of any sequence of SEQ ID NO: 1-14,
(b) a polypeptide encoded by an isolated polynucleotide comprising a polynucleotide sequence which has at least 85% identity, preferably at least 90% identity, more preferably at least 95% identity, even more preferably at least 97-99% or exact identity, to any sequence of SEQ ID NO: 15 over the entire length of the selected sequence of SEQ ID NO: 15; or
(c) a polypeptide encoded by an isolated polynucleotide comprising a polynucleotide sequence encoding a polypeptide which has at least 85% identity, preferably at least 90% identity, more preferably at least 95% identity, even more preferably at least 97-99% or exact identity, to the amino acid sequence of any sequence of SEQ ID NO: 1-14.

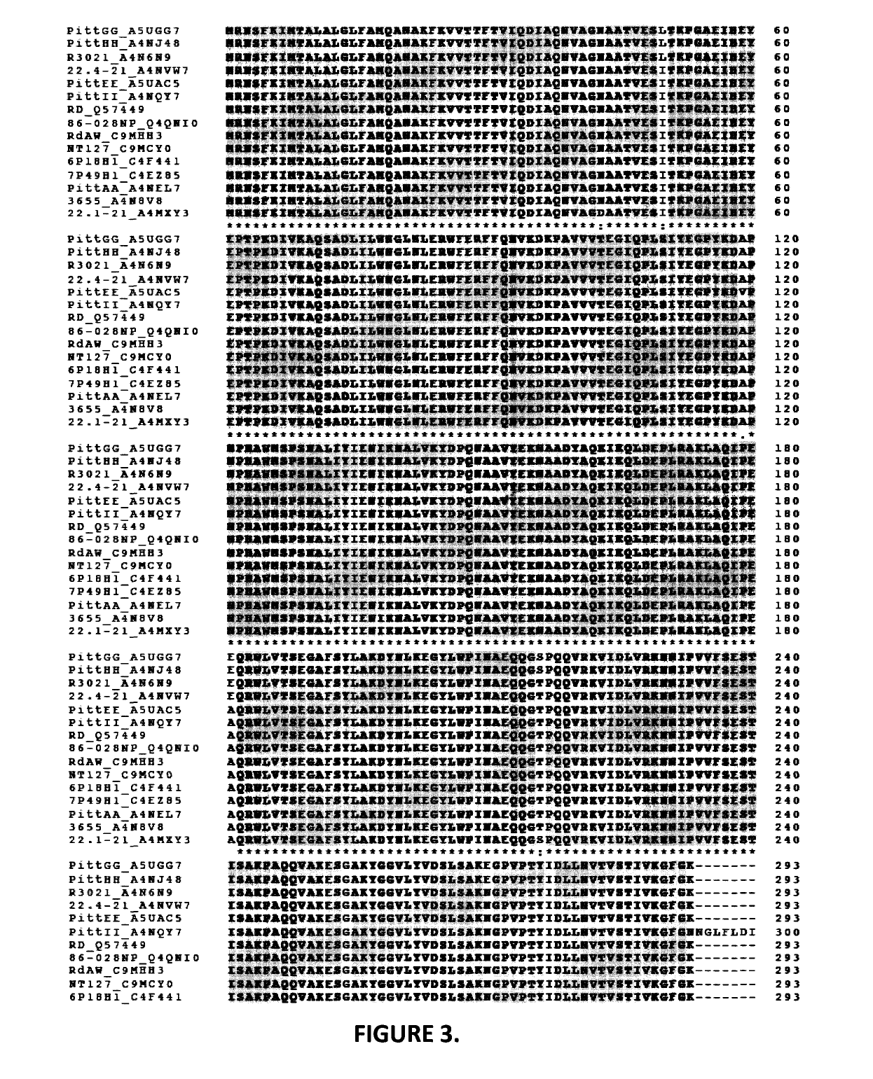

The Protein F polypeptides provided in SEQ ID NO: 1-14 are the Protein F polypeptides from non typeable H. influenzae strains. Further Protein F sequences have been ascertained from H. influenzae strains listed in FIG. 3

The invention also provides an immunogenic fragment of a Protein F polypeptide, that is, a contiguous portion of the Protein F polypeptide which has the same or substantially the same immunogenic activity as the polypeptide comprising the corresponding amino acid sequence selected from SEQ ID NO: 1-14. That is to say, the fragment (if necessary when coupled to a carrier) is capable of raising an immune response which recognises the Protein F polypeptide. Such an immunogenic fragment may include, for example, the Protein F polypeptide lacking an N-terminal leader sequence or parts thereof, and/or a transmembrane domain and/or a C-terminal anchor domain. In a preferred aspect the immunogenic fragment of Protein F according to the invention comprises substantially all of the extracellular domain of a polypeptide which has at least 85% identity, preferably at least 90% identity, more preferably at least 95% identity, most preferably at least 97-99% identity, to that a sequence selected from SEQ ID NO: 1-14 over the entire length of said sequence.

A fragment is a polypeptide having an amino acid sequence that is entirely the same as part but not all of any amino acid sequence of any polypeptide of the invention. As with Protein F polypeptides, fragments may be “free-standing,” or comprised within a larger polypeptide of which they form a part or region, most preferably as a single continuous region in a single larger polypeptide. A fragment may therefore be shorter than the full-length native sequence, or, if comprised within a larger polypeptide, may be a full length native sequence or a longer fusion protein.

Preferred fragments include, for example, truncation polypeptides having a portion of an amino acid sequence selected from SEQ ID NO: 1-14 or of variants thereof, such as a continuous series of residues that includes an amino- and/or carboxyl-terminal amino acid sequence. Degradation forms of the polypeptides of the invention produced by or in a host cell, are also preferred. Further preferred are fragments characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions.

Further preferred fragments include an isolated polypeptide comprising an amino acid sequence having at least 15, 20, 30, 40, 50 or 100 contiguous amino acids from an amino acid sequence selected from SEQ ID NO: 1-14 or an isolated polypeptide comprising an amino acid sequence having at least 15, 20, 30, 40, 50 or 100 contiguous amino acids truncated or deleted from an amino acid sequence selected from SEQ ID NO: 1-14.

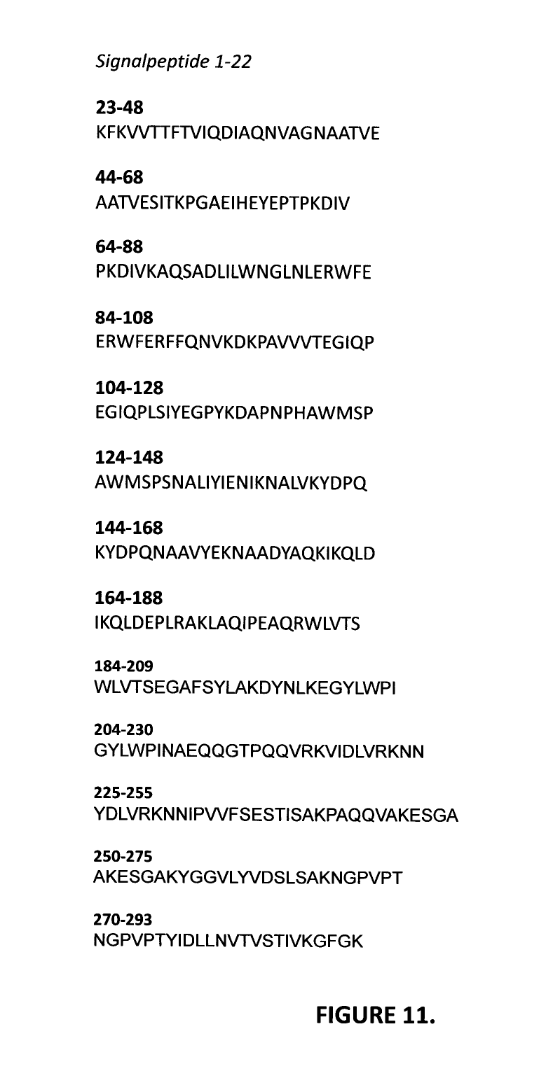

Still further preferred fragments are those which comprise a B-cell epitope, for example those fragments/peptides described in FIG. 11.

Fragments of the full length protein F of the invention may be employed for producing the corresponding full-length polypeptide by peptide synthesis; therefore, these fragments may be employed as intermediates for producing the full-length protein F or other polypeptides based upon the protein F sequence of the invention.

Particularly preferred are variants in which several, 5-10, 1-5, 1-3, 1-2 or 1 amino acids are substituted, deleted, or added in any combination.

The polypeptides, or immunogenic fragments, of the invention may be in the form of the “mature” protein or may be a part of a larger protein such as a precursor or a fusion protein. It is often advantageous to include an additional amino acid sequence which contains secretory or leader sequences, pro-sequences, sequences which aid in purification such as multiple histidine residues, or an additional sequence for stability during recombinant production. Furthermore, addition of exogenous polypeptide or lipid tail or polynucleotide sequences to increase the immunogenic potential of the final molecule is also considered.

In one aspect, the invention relates to genetically engineered soluble fusion proteins comprising a polypeptide of the present invention, or a fragment thereof, and various portions of the constant regions of heavy or light chains of immunoglobulins of various subclasses (IgG, IgM, IgA, IgE).

Preferred as an immunoglobulin is the constant part of the heavy chain of human IgG, particularly IgG1, where fusion takes place at the hinge region. particular embodiment, the Fc part can be removed simply by incorporation of a cleavage sequence which can be cleaved with blood clotting factor Xa.

Furthermore, this invention relates to processes for the preparation of these fusion proteins by genetic engineering, and to the use thereof for drug screening, diagnosis and therapy. A further aspect of the invention also relates to polynucleotides encoding such fusion proteins. Examples of fusion protein technology can be found in WO94/29458 and WO94/22914.

The proteins may be chemically conjugated, or expressed as recombinant fusion proteins allowing increased levels to be produced in an expression system as compared to non-fused protein. The fusion partner may assist in providing T helper epitopes (immunological fusion partner), preferably T helper epitopes recognised by humans, or assist in expressing the protein (expression enhancer) at higher yields than the native recombinant protein. Preferably the fusion partner will be both an immunological fusion partner and expression enhancing partner.

Fusion partners include protein D from Haemophilus influenzae (EP 594610), protein E from Haemophilus influenzae (EP 1 973 933) and/or the non-structural protein from influenza virus, NS1 (hemagglutinin). Another fusion partner is the protein known as Omp26 (WO 97/01638). Another fusion partner is the protein known as LytA. Preferably the C terminal portion of the molecule is used. LytA is derived from Streptococcus pneumoniae which synthesize an N-acetyl-L-alanine amidase, amidase LytA, (coded by the lytA gene {Gene, 43 (1986) page 265-272}) an autolysin that specifically degrades certain bonds in the peptidoglycan backbone. The C-terminal domain of the LytA protein is responsible for the affinity to the choline or to some choline analogues such as DEAE. This property has been exploited for the development of E. coli C-LytA expressing plasmids useful for expression of fusion proteins. Purification of hybrid proteins containing the C-LytA fragment at its amino terminus has been described {Biotechnology: 10, (1992) page 795-798}. It is possible to use the repeat portion of the LytA molecule found in the C terminal end starting at residue 178, for example residues 188-305.

The present invention also includes variants of the aforementioned protein F and peptides, that is peptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr.

Polypeptides of the present invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

It is most preferred that a polypeptide of the invention is derived from non typeable H. influenzae, however, it may preferably be obtained from other organisms of the same taxonomic genus. A polypeptide of the invention may also be obtained, for example, from organisms of the same taxonomic family or order.

### Polynucleotides

It is an object of the invention to provide polynucleotides that encode Protein F polypeptides, particularly polynucleotides that encode the polypeptides herein designated Protein F.

In a particularly preferred embodiment of the invention the polynucleotides comprise a region encoding Protein F polypeptides comprising sequences set out in SEQ ID NO: 15 which include full length gene, or a variant thereof.

The Protein F polynucleotides provided in SEQ ID NO: 15 are the Protein F polynucleotides from non typeable H. influenzae strain 3655. Other sequences have been determined of genes encoding protein F from H. influenzae strains listed in FIG. 3.

As a further aspect of the invention there are provided isolated nucleic acid molecules encoding and/or expressing Protein F polypeptides and polynucleotides, particularly non typeable H. influenzae Protein F polypeptides and polynucleotides, including, for example, unprocessed RNAs, ribozyme RNAs, mRNAs, cDNAs, genomic DNAs, B- and Z-DNAs. Further embodiments of the invention include biologically, diagnostically, prophylactically, clinically or therapeutically useful polynucleotides and polypeptides, and variants thereof, and compositions comprising the same.

Another aspect of the invention relates to isolated polynucleotides, including at least one full length gene, that encodes a Protein F polypeptide having a deduced amino acid sequence of SEQ ID NO: 1-14 and polynucleotides closely related thereto and variants thereof.

In another particularly preferred embodiment of the invention relates to Protein F polypeptide from non typeable H. influenzae comprising or consisting of an amino acid sequence selected from SEQ ID NO: 1-14 or a variant thereof.

Using the information provided herein, such as a polynucleotide sequences set out in SEQ ID NO: 15, a polynucleotide of the invention encoding Protein F polypeptides may be obtained using standard cloning and screening methods, such as those for cloning and sequencing chromosomal DNA fragments from bacteria using non typeable H. influenzae strain 3224A (or 3655) cells as starting material, followed by obtaining a full length clone.

Moreover, each DNA sequence set out in SEQ ID NO: 15 contains an open reading frame encoding a protein having about the number of amino acid residues set forth in SEQ ID NO: 1 with a deduced molecular weight that can be calculated using amino acid residue molecular weight values well known to those skilled in the art.

The polynucleotides of SEQ ID NO: 15, between the start codon and the stop codon, encode respectively the polypeptide of SEQ ID NO: 1.

In a further aspect, the present invention provides for an isolated polynucleotide comprising or consisting of:

(a) a polynucleotide sequence which has at least 85% identity, preferably at least 90% identity, more preferably at least 95% identity, even more preferably at least 97-99% or exact identity, to any polynucleotide sequence from SEQ ID NO: 15 over the entire length of the polynucleotide sequence from SEQ ID NO: 15; or
(b) a polynucleotide sequence encoding a polypeptide which has at least 85% identity, preferably at least 90% identity, more preferably at least 95% identity, even more preferably at least 97-99% or 100% exact identity, to any amino acid sequence selected from SEQ ID NO: 1-14 (or fragment thereof), over the entire length of the amino acid sequence from SEQ ID NO: 1-14 (or fragment thereof).

A polynucleotide encoding a polypeptide of the present invention, including homologs and orthologs from species other than non typeable H. influenzae, may be obtained by a process which comprises the steps of screening an appropriate library under stringent hybridization conditions (for example, using a temperature in the range of 45-65° C. and an SDS concentration from 0.1-1%) with a labeled or detectable probe consisting of or comprising any sequence selected from SEQ ID NO: 15 or a fragment thereof; and isolating a full-length gene and/or genomic clones containing said polynucleotide sequence.

The invention provides a polynucleotide sequence identical over its entire length to a coding sequence (open reading frame) set out in SEQ ID NO: 15.

Also provided by the invention is a coding sequence for a mature polypeptide or a fragment thereof, by itself as well as a coding sequence for a mature polypeptide or a fragment in reading frame with another coding sequence, such as a sequence encoding a leader or secretory sequence, a pre-, or pro- or prepro-protein sequence. The polynucleotide of the invention may also contain at least one non-coding sequence, including for example, but not limited to at least one non-coding 5′ and 3′ sequence, such as the transcribed but non-translated sequences, termination signals (such as rho-dependent and rho-independent termination signals), ribosome binding sites, Kozak sequences, sequences that stabilize mRNA, introns, and polyadenylation signals. The polynucleotide sequence may also comprise additional coding sequence encoding additional amino acids. For example, a marker sequence that facilitates purification of the fused polypeptide can be encoded. In certain embodiments of the invention, the marker sequence is a hexa-histidine peptide, as provided in the pQE vector (Qiagen, Inc.) and described in Gentz et al., Proc. Natl. Acad. Sci., USA 86: 821-824 (1989), or an HA peptide tag (Wilson et al., Cell 37: 767 (1984), both of which may be useful in purifying polypeptide sequence fused to them. Polynucleotides of the invention also include, but are not limited to, polynucleotides comprising a structural gene and its naturally associated sequences that control gene expression.

The nucleotide sequence encoding the Protein F polypeptide of SEQ ID NO: 1-14 may be identical to the corresponding polynucleotide encoding sequence of SEQ ID NO: 15 (or comprised within SEQ ID NO: 15). Alternatively it may be any sequence, which as a result of the redundancy (degeneracy) of the genetic code, also encodes a polypeptide of SEQ ID NO: 1-14.

The term “polynucleotide encoding a polypeptide” as used herein encompasses polynucleotides that include a sequence encoding a polypeptide of the invention, particularly a bacterial polypeptide and more particularly a polypeptide of the non typeable H. influenzae Protein F having an amino acid sequence set out in any of the sequences of SEQ ID NO: 1-14 or fragments thereof. The term also encompasses polynucleotides that include a single continuous region or discontinuous regions encoding the polypeptide (for example, polynucleotides interrupted by integrated phage, an integrated insertion sequence, an integrated vector sequence, an integrated transposon sequence, or due to RNA editing or genomic DNA reorganization) together with additional regions, that also may contain coding and/or non-coding sequences.

The invention further relates to variants of the polynucleotides described herein that encode variants of a polypeptide having a deduced amino acid sequence of any of the sequences of SEQ ID NO: 1-14. Fragments of polynucleotides of the invention may be used, for example, to synthesize full-length polynucleotides of the invention.

Preferred fragments are those polynucleotides which encode a B-cell epitope, for example the fragments/peptides described in FIG. 11, and recombinant, chimeric genes comprising said polynucleotide fragments.

Further particularly preferred embodiments are polynucleotides encoding Protein F variants, that have the amino acid sequence of Protein F polypeptide of any sequence from SEQ ID NO: 1-14 in which several, a few, 5 to 10, 1 to 5, 1 to 3, 2, 1 or no amino acid residues are substituted, modified, deleted and/or added, in any combination. Especially preferred among these are silent substitutions, additions and deletions, that do not alter the properties and activities of Protein F polypeptide (for instance those properties described in the Example section herein).

Further preferred embodiments of the invention are polynucleotides that are at least 85% identical over their entire length to polynucleotides encoding Protein E polypeptides having an amino acid sequence set out in any of the sequences of SEQ ID NO: 1-14, and polynucleotides that are complementary to such polynucleotides. Alternatively, most highly preferred are polynucleotides that comprise a region that is at least 90% identical over its entire length to polynucleotides encoding Protein F polypeptides and polynucleotides complementary thereto. In this regard, polynucleotides at least 95% identical over their entire length to the same are particularly preferred. Furthermore, those with at least 97% are highly preferred among those with at least 95%, and among these those with at least 98% and at least 99% are particularly highly preferred, with at least 99% being the more preferred.

Preferred embodiments are polynucleotides encoding polypeptides that retain substantially the same biological function or activity as the mature polypeptide encoded by a DNA sequence selected from SEQ ID NO: 15.

In accordance with certain preferred embodiments of this invention there are provided polynucleotides that hybridize, particularly under stringent conditions, to Protein F polynucleotide sequences, such as those polynucleotides of SEQ ID NO: 15.

The invention further relates to polynucleotides that hybridize to the polynucleotide sequences provided herein. In this regard, the invention especially relates to polynucleotides that hybridize under stringent conditions to the polynucleotides described herein. As herein used, the terms “stringent conditions” and “stringent hybridization conditions” mean hybridization occurring only if there is at least 95% and preferably at least 97% identity between the sequences. A specific example of stringent hybridization conditions is overnight incubation at 42° C. in a solution comprising: 50% formamide, 5×SSC (150 mM NaCl, 15 mM trisodium citrate), 50 mM sodium phosphate (pH7.6), 5×Denhardt's solution, 10% dextran sulfate, and 20 micrograms/ml of denatured, sheared salmon sperm DNA, followed by washing the hybridization support in 0.1×SSC at about 65° C. Hybridization and wash conditions are well known and exemplified in Sambrook, et al., Molecular Cloning: A Laboratory Manual, Second Edition, Cold Spring Harbor, N.Y., (1989), particularly Chapter 11 therein. Solution hybridization may also be used with the polynucleotide sequences provided by the invention.

The invention also provides a polynucleotide consisting of or comprising a polynucleotide sequence obtained by screening an appropriate library containing the complete gene for a polynucleotide sequence set forth in any of the sequences of SEQ ID NO: 15 under stringent hybridization conditions with a probe having the sequence of said polynucleotide sequence set forth in the corresponding sequence of SEQ ID NO: 15 or a fragment thereof; and isolating said polynucleotide sequence. Fragments useful for obtaining such a polynucleotide include, for example, probes and primers fully described elsewhere herein.

As discussed elsewhere herein regarding polynucleotide assays of the invention, for instance, the polynucleotides of the invention may be used as a hybridization probe for RNA, cDNA and genomic DNA to isolate full-length cDNAs and genomic clones encoding Protein F and to isolate cDNA and genomic clones of other genes that have a high identity, particularly high sequence identity, to the Protein F genes. Such probes generally will comprise at least 15 nucleotide residues or base pairs. Preferably, such probes will have at least 30 nucleotide residues or base pairs and may have at least 50 nucleotide residues or base pairs. Particularly preferred probes will have at least 20 nucleotide residues or base pairs and will have less than 30 nucleotide residues or base pairs.

A coding region of Protein F genes may be isolated by screening using a DNA sequence provided in SEQ ID NO: 15 to synthesize an oligonucleotide probe. A labeled oligonucleotide having a sequence complementary to that of a gene of the invention is then used to screen a library of cDNA, genomic DNA or mRNA to determine which members of the library the probe hybridizes to.

There are several methods available and well known to those skilled in the art to obtain full-length DNAs, or extend short DNAs, for example those based on the method of Rapid Amplification of cDNA ends (RACE) (see, for example, Frohman, et al., PNAS USA 85: 8998-9002, 1988). Recent modifications of the technique, exemplified by the Marathon™ technology (Clontech Laboratories Inc.) for example, have significantly simplified the search for longer cDNAs. In the Marathon™ technology, cDNAs have been prepared from mRNA extracted from a chosen tissue and an ‘adaptor’ sequence ligated onto each end. Nucleic acid amplification (PCR) is then carried out to amplify the “missing” 5′ end of the DNA using a combination of gene specific and adaptor specific oligonucleotide primers. The PCR reaction is then repeated using “nested” primers, that is, primers designed to anneal within the amplified product (typically an adaptor specific primer that anneals further 3′ in the adaptor sequence and a gene specific primer that anneals further 5′ in the selected gene sequence). The products of this reaction can then be analyzed by DNA sequencing and a full-length DNA constructed either by joining the product directly to the existing DNA to give a complete sequence, or carrying out a separate full-length PCR using the new sequence information for the design of the 5′ primer.

The polynucleotides and polypeptides of the invention may be employed, for example, as research reagents and materials for discovery of treatments of and diagnostics for diseases, particularly human diseases, as further discussed herein relating to polynucleotide assays.

The polynucleotides of the invention that are oligonucleotides derived from a sequence of SEQ ID NO: 15 may be used in the processes herein as described, but preferably for PCR, to determine whether or not the polynucleotides identified herein in whole or in part are transcribed in bacteria in infected tissue. It is recognized that such sequences will also have utility in diagnosis of the stage of infection and type of infection the pathogen has attained.

The invention also provides polynucleotides that encode a polypeptide that is the mature protein plus additional amino or carboxyl-terminal amino acids, or amino acids interior to the mature polypeptide (when the mature form has more than one polypeptide chain, for instance). Such sequences may play a role in processing of a protein from precursor to a mature form, may allow protein transport, may lengthen or shorten protein half-life or may facilitate manipulation of a protein for assay or production, among other things. As generally is the case in vivo, the additional amino acids may be processed away from the mature protein by cellular enzymes.

For each and every polynucleotide of the invention there is provided a polynucleotide complementary to it. It is preferred that these complementary polynucleotides are fully complementary to each polynucleotide with which they are complementary.

A precursor protein, having a mature form of the polypeptide fused to one or more prosequences may be an inactive form of the polypeptide. When prosequences are removed such inactive precursors generally are activated. Some or all of the prosequences may be removed before activation. Generally, such precursors are called proproteins.

In addition to the standard A, G, C, T/U representations for nucleotides, the term “N” may also be used in describing certain polynucleotides of the invention. “N” means that any of the four DNA or RNA nucleotides may appear at such a designated position in the DNA or RNA sequence, except it is preferred that N is not a nucleic acid that when taken in combination with adjacent nucleotide positions, when read in the correct reading frame, would have the effect of generating a premature termination codon in such reading frame.

In sum, a polynucleotide of the invention may encode a mature protein, a mature protein plus a leader sequence (which may be referred to as a preprotein), a precursor of a mature protein having one or more prosequences that are not the leader sequences of a preprotein, or a preproprotein, which is a precursor to a proprotein, having a leader sequence and one or more prosequences, which generally are removed during processing steps that produce active and mature forms of the polypeptide.

In accordance with an aspect of the invention, there is provided the use of a polynucleotide of the invention for therapeutic or prophylactic purposes, in particular genetic immunization.

The use of a polynucleotide of the invention in genetic immunization will preferably employ a suitable delivery method such as direct injection of plasmid DNA into muscles (Wolff et al., Hum Mol Genet (1992) 1: 363, Manthorpe et al., Hum. Gene Ther. (1983) 4: 419), delivery of DNA complexed with specific protein carriers (Wu et al., J Biol Chem. (1989) 264: 16985), coprecipitation of DNA with calcium phosphate (Benvenisty & Reshef, PNAS USA, (1986) 83: 9551), encapsulation of DNA in various forms of liposomes (Kaneda et al., Science (1989) 243: 375), particle bombardment (Tang et al., Nature (1992) 356:152, Eisenbraun et al., DNA Cell Biol (1993) 12: 791) and in vivo infection using cloned retroviral vectors (Seeger et al., PNAS USA (1984) 81: 5849).

### Vectors, Host Cells, Expression Systems

The invention also relates to vectors that comprise a polynucleotide or polynucleotides of the invention, host cells that are genetically engineered with vectors of the invention and the production of polypeptides of the invention by recombinant techniques. Cell-free translation systems can also be employed to produce such proteins using RNAs derived from the DNA constructs of the invention.

Recombinant polypeptides of the present invention may be prepared by processes well known in those skilled in the art from genetically engineered host cells comprising expression systems. Accordingly, in a further aspect, the present invention relates to expression systems that comprise a polynucleotide or polynucleotides of the present invention, to host cells which are genetically engineered with such expression systems, and to the production of polypeptides of the invention by recombinant techniques.

For recombinant production of the polypeptides of the invention, host cells can be genetically engineered to incorporate expression systems or portions thereof or polynucleotides of the invention. Introduction of a polynucleotide into the host cell can be effected by methods described in many standard laboratory manuals, such as Davis, et al., BASIC METHODS IN MOLECULAR BIOLOGY, (1986) and Sambrook, et al., MOLECULAR CLONING: A LABORATORY MANUAL, 2nd Ed., Cold Spring Harbor Laboratory Press, Cold Spring Harbor, N.Y. (1989), such as, calcium phosphate transfection, DEAE-dextran mediated transfection, transvection, microinjection, cationic lipid-mediated transfection, electroporation, conjugation, transduction, scrape loading, ballistic introduction and infection.

Representative examples of appropriate hosts include bacterial cells, such as cells of streptococci, staphylococci, enterococci, E. coli, streptomyces, cyanobacteria, Bacillus subtilis, Neisseria meningitidis, Haemophilus influenzae and Moraxella catarrhalis; fungal cells, such as cells of a yeast, Kluveromyces, Saccharomyces, Pichia, a basidiomycete, Candida albicans and Aspergillus; insect cells such as cells of Drosophila S2 and Spodoptera Sf9; animal cells such as CHO, COS, HeLa, C127, 3T3, BHK, 293, CV-1 and Bowes melanoma cells; and plant cells, such as cells of a gymnosperm or angiosperm.

A great variety of expression systems can be used to produce the polypeptides of the invention. Such vectors include, among others, chromosomal-, episomal- and virus-derived vectors, for example, vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses, picornaviruses, retroviruses, and alphaviruses and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., MOLECULAR CLONING, A LABORATORY MANUAL, (supra).

In recombinant expression systems in eukaryotes, for secretion of a translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, ion metal affinity chromatography (IMAC) is employed for purification. Well known techniques for refolding proteins may be employed to regenerate active conformation when the polypeptide is denatured during intracellular synthesis, isolation and or purification.

The expression system may also be a recombinant live microorganism, such as a virus or bacterium. The gene of interest can be inserted into the genome of a live recombinant virus or bacterium. Inoculation and in vivo infection with this live vector will lead to in vivo expression of the antigen and induction of immune responses. Viruses and bacteria used for this purpose are for instance: poxviruses (e.g; vaccinia, fowlpox, canarypox), alphaviruses (Sindbis virus, Semliki Forest Virus, Venezuelian Equine Encephalitis Virus), adenoviruses, adeno-associated virus, picornaviruses (poliovirus, rhinovirus), herpesviruses (varicella zoster virus, etc), Listeria, Salmonella, Shigella, BCG, streptococci. These viruses and bacteria can be virulent, or attenuated in various ways in order to obtain live vaccines. Such live vaccines also form part of the invention.

### Diagnostic, Prognostic, Serotyping and Mutation Assays

This invention is also related to the use of Protein F polynucleotides and polypeptides of the invention for use as diagnostic reagents. Detection of Protein F polynucleotides and/or polypeptides in a eukaryote, particularly a mammal, and especially a human, will provide a diagnostic method for diagnosis of disease, staging of disease or response of an infectious organism to drugs. Eukaryotes, particularly mammals, and especially humans, particularly those infected or suspected to be infected with an organism comprising the Protein F genes or proteins, may be detected at the nucleic acid or amino acid level by a variety of well known techniques as well as by methods provided herein.

Polypeptides and polynucleotides for prognosis, diagnosis or other analysis may be obtained from a putatively infected and/or infected individual's bodily materials. Polynucleotides from any of these sources, particularly DNA or RNA, may be used directly for detection or may be amplified enzymatically by using PCR or any other amplification technique prior to analysis. RNA, particularly mRNA, cDNA and genomic DNA may also be used in the same ways. Using amplification, characterization of the species and strain of infectious or resident organism present in an individual, may be made by an analysis of the genotype of a selected polynucleotide of the organism. Deletions and insertions can be detected by a change in size of the amplified product in comparison to a genotype of a reference sequence selected from a related organism, preferably a different species of the same genus or a different strain of the same species. Point mutations can be identified by hybridizing amplified DNA to labeled Protein F polynucleotide sequences. Perfectly or significantly matched sequences can be distinguished from imperfectly or more significantly mismatched duplexes by DNase or RNase digestion, for DNA or RNA respectively, or by detecting differences in melting temperatures or renaturation kinetics. Polynucleotide sequence differences may also be detected by alterations in the electrophoretic mobility of polynucleotide fragments in gels as compared to a reference sequence. This may be carried out with or without denaturing agents. Polynucleotide differences may also be detected by direct DNA or RNA sequencing. See, for example, Myers et al., Science, 230: 1242 (1985). Sequence changes at specific locations also may be revealed by nuclease protection assays, such as RNase, V1 and S1 protection assay or a chemical cleavage method. See, for example, Cotton et al., Proc. Natl. Acad. Sci., USA, 85: 4397-4401 (1985).

In another embodiment, an array of oligonucleotides probes comprising Protein F nucleotide sequences or fragments thereof can be constructed to conduct efficient screening of, for example, genetic mutations, serotype, taxonomic classification or identification. Array technology methods are well known and have general applicability and can be used to address a variety of questions in molecular genetics including gene expression, genetic linkage, and genetic variability (see, for example, Chee et al., Science, 274: 610 (1996)).

Thus in another aspect, the present invention relates to a diagnostic kit which comprises:

(a) a polynucleotide of the present invention, preferably any of the nucleotide sequences of SEQ ID NO: 15, or a fragment thereof;
(b) a nucleotide sequence complementary to that of (a);
(c) a polypeptide of the present invention, preferably any of the polypeptides of SEQ ID NO: 1-14 or a fragment thereof; or
(d) an antibody to a polypeptide of the present invention, preferably to any of the polypeptides of SEQ ID NO: 1-14.

It will be appreciated that in any such kit, (a), (b), (c) or (d) may comprise a substantial component. Such a kit will be of use in diagnosing a disease or susceptibility to a disease, among others.

This invention also relates to the use of polynucleotides of the present invention as diagnostic reagents. Detection of a mutated form of a polynucleotide of the invention, preferably any sequence of SEQ ID NO: 15, which is associated with a disease or pathogenicity will provide a diagnostic tool that can add to, or define, a diagnosis of a disease, a prognosis of a course of disease, a determination of a stage of disease, or a susceptibility to a disease, which results from under-expression, over-expression or altered expression of the polynucleotide. Organisms, particularly infectious organisms, carrying mutations in such polynucleotide may be detected at the polynucleotide level by a variety of techniques, such as those described elsewhere herein.

Cells from an organism carrying mutations or polymorphisms (allelic variations) in a polynucleotide and/or polypeptide of the invention may also be detected at the polynucleotide or polypeptide level by a variety of techniques, to allow for serotyping, for example. For example, RT-PCR can be used to detect mutations in the RNA. It is particularly preferred to use RT-PCR in conjunction with automated detection systems, such as, for example, GeneScan. RNA, cDNA or genomic DNA may also be used for the same purpose, PCR. As an example, PCR primers complementary to a polynucleotide encoding Protein F polypeptides can be used to identify and analyze mutations.

The invention further provides primers with 1, 2, 3 or 4 nucleotides removed from the 5′ and/or the 3′ end. These primers may be used for, among other things, amplifying Protein F DNA and/or RNA isolated from a sample derived from an individual, such as a bodily material. The primers may be used to amplify a polynucleotide isolated from an infected individual, such that the polynucleotide may then be subject to various techniques for elucidation of the polynucleotide sequence. In this way, mutations in the polynucleotide sequence may be detected and used to diagnose and/or prognose the infection or its stage or course, or to serotype and/or classify the infectious agent.

The invention further provides a process for diagnosing disease, preferably bacterial infections, more preferably infections caused by non typeable H. influenzae, comprising determining from a sample derived from an individual, such as a bodily material, an increased level of expression of polynucleotide having a sequence of any of the sequences of SEQ ID NO: 15. Increased or decreased expression of Protein F polynucleotide can be measured using any one of the methods well known in the art for the quantitation of polynucleotides, such as, for example, amplification, PCR, RT-PCR, RNase protection, Northern blotting, spectrometry and other hybridization methods.

In addition, a diagnostic assay in accordance with the invention for detecting over-expression of Protein F polypeptides compared to normal control tissue samples may be used to detect the presence of an infection, for example.

Assay techniques that can be used to determine levels of Protein F polypeptides, in a sample derived from a host, such as a bodily material, are well-known to those of skill in the art. Such assay methods include radio-immunoassays, competitive-binding assays, Western Blot analysis, antibody sandwich assays, antibody detection and ELISA assays.

The polynucleotides of the invention may be used as components of polynucleotide arrays, preferably high density arrays or grids. These high density arrays are particularly useful for diagnostic and prognostic purposes. For example, a set of spots each comprising a different gene, and further comprising a polynucleotide or polynucleotides of the invention, may be used for probing, such as using hybridization or nucleic acid amplification, using a probe obtained or derived from a bodily sample, to determine the presence of a particular polynucleotide sequence or related sequence in an individual. Such a presence may indicate the presence of a pathogen, particularly non-typeable H. influenzae, and may be useful in diagnosing and/or prognosing disease or a course of disease. A grid comprising a number of variants of any polynucleotide sequence of SEQ ID NO: 15 is preferred. Also preferred are a number of variants of a polynucleotide sequence encoding any polypeptide sequence of SEQ ID NO: 1-14.

### Antibodies

The polypeptides and polynucleotides of the invention or variants thereof, or cells expressing the same can be used as immunogens to produce antibodies immunospecific for such polypeptides or polynucleotides respectively. Alternatively, mimotopes, particularly peptide mimotopes, of epitopes within the polypeptide sequence may also be used as immunogens to produce antibodies immunospecific for the polypeptide of the invention. The term “immunospecific” means that the antibodies have substantially greater affinity for the polypeptides of the invention than their affinity for other related polypeptides in the prior art.

In certain preferred embodiments of the invention there are provided antibodies against Protein F polypeptides or polynucleotides.

Antibodies generated against the polypeptides or polynucleotides of the invention can be obtained by administering the polypeptides and/or polynucleotides of the invention, or epitope-bearing fragments of either or both, analogues of either or both, or cells expressing either or both, to an animal, preferably a nonhuman, using routine protocols. For preparation of monoclonal antibodies, any technique known in the art that provides antibodies produced by continuous cell line cultures can be used. Examples include various techniques, such as those in Kohler, G. and Milstein, C., Nature 256: 495-497 (1975); Kozbor et al., Immunology Today 4: 72 (1983); Cole et al., pg. 77-96 in MONOCLONAL ANTIBODIES AND CANCER THERAPY, Alan R. Liss, Inc. (1985).

Techniques for the production of single chain antibodies (U.S. Pat. No. 4,946,778) can be adapted to produce single chain antibodies to polypeptides or polynucleotides of this invention. Also, transgenic mice, or other organisms or animals, such as other mammals, may be used to express humanized antibodies immunospecific to the polypeptides or polynucleotides of the invention.

Alternatively, phage display technology may be utilized to select antibody genes with binding activities towards a polypeptide of the invention either from repertoires of PCR amplified v-genes of lymphocytes from humans screened for possessing anti-Protein F or from naive libraries (McCafferty, et al., (1990), Nature 348, 552-554; Marks, et al., (1992) Biotechnology 10, 779-783). The affinity of these antibodies can also be improved by, for example, chain shuffling (Clackson et al., (1991) Nature 352: 628).

The above-described antibodies may be employed to isolate or to identify clones expressing the polypeptides or polynucleotides of the invention to purify the polypeptides or polynucleotides by, for example, affinity chromatography.

Thus, among others, antibodies against Protein F polypeptides or Protein F polynucleotides may be employed to treat infections, particularly bacterial infections.

Polypeptide variants include antigenically, epitopically or immunologically equivalent variants form a particular aspect of this invention.

Preferably, the antibody or variant thereof is modified to make it less immunogenic in the individual. For example, if the individual is human the antibody may most preferably be “humanized,” where the complimentarity determining region or regions of the hybridoma-derived antibody has been transplanted into a human monoclonal antibody, for example as described in Jones et al. (1986), Nature 321, 522-525 or Tempest et al., (1991) Biotechnology 9, 266-273.

### Antagonists and Agonists—Assays and Molecules

Polypeptides and polynucleotides of the invention may also be used to assess the binding of small molecule substrates and ligands in, for example, cells, cell-free preparations, chemical libraries, and natural product mixtures. These substrates and ligands may be natural substrates and ligands or may be structural or functional mimetics. See, e.g., Coligan et al., Current Protocols in Immunology 1(2): Chapter 5 (1991).

The screening methods may simply measure the binding of a candidate compound to the polypeptide or polynucleotide, or to cells or membranes bearing the polypeptide or polynucleotide, or a fusion protein of the polypeptide by means of a label directly or indirectly associated with the candidate compound. Alternatively, the screening method may involve competition with a labeled competitor. Further, these screening methods may test whether the candidate compound results in a signal generated by activation or inhibition of the polypeptide or polynucleotide, using detection systems appropriate to the cells comprising the polypeptide or polynucleotide. Inhibitors of activation are generally assayed in the presence of a known agonist and the effect on activation by the agonist by the presence of the candidate compound is observed. Constitutively active polypeptide and/or constitutively expressed polypeptides and polynucleotides may be employed in screening methods for inverse agonists or inhibitors, in the absence of an agonist or inhibitor, by testing whether the candidate compound results in inhibition of activation of the polypeptide or polynucleotide, as the case may be. Further, the screening methods may simply comprise the steps of mixing a candidate compound with a solution containing a polypeptide or polynucleotide of the present invention, to form a mixture, measuring Protein F polypeptides and/or polynucleotides activity in the mixture, and comparing the Protein F polypeptides and/or polynucleotides activity of the mixture to a standard. Fusion proteins, such as those made from Fc portion and Protein F polypeptides, as hereinbefore described, can also be used for high-throughput screening assays to identify antagonists of the polypeptide of the present invention, as well as of phylogenetically and and/or functionally related polypeptides (see D. Bennett et al., J Mol Recognition, 8:52-58 (1995); and K. Johanson et al., J Biol Chem, 270(16):9459-9471 (1995)).

The polynucleotides, polypeptides and antibodies that bind to and/or interact with a polypeptide of the present invention may also be used to configure screening methods for detecting the effect of added compounds on the production of mRNA and/or polypeptide in cells. For example, an ELISA assay may be constructed for measuring secreted or cell associated levels of polypeptide using monoclonal and polyclonal antibodies by standard methods known in the art. This can be used to discover agents which may inhibit or enhance the production of polypeptide (also called antagonist or agonist, respectively) from suitably manipulated cells or tissues.

The invention also provides a method of screening compounds to identify those which enhance (agonist) or block (antagonist) the action of Protein F polypeptides or polynucleotides, particularly those compounds that are bacteriostatic and/or bactericidal. The method of screening may involve high-throughput techniques. For example, to screen for agonists or antagonists, a synthetic reaction mix, a cellular compartment, such as a membrane, cell envelope or cell wall, or a preparation of any thereof, comprising Protein F polypeptides and a labeled substrate or ligand of such polypeptide is incubated in the absence or the presence of a candidate molecule that may be a Protein F agonist or antagonist. The ability of the candidate molecule to agonize or antagonize the Protein F polypeptide is reflected in decreased binding of the labeled ligand or decreased production of product from such substrate. Molecules that bind gratuitously, i.e., without inducing the effects of Protein F polypeptide are most likely to be good antagonists. Molecules that bind well and, as the case may be, increase the rate of product production from substrate, increase signal transduction, or increase chemical channel activity are agonists. Detection of the rate or level of, as the case may be, production of product from substrate, signal transduction, or chemical channel activity may be enhanced by using a reporter system. Reporter systems that may be useful in this regard include but are not limited to colorimetric, labeled substrate converted into product, a reporter gene that is responsive to changes in Protein F polynucleotide or polypeptide activity, and binding assays known in the art.

Another example of an assay for Protein F agonists is a competitive assay that combines Protein F and a potential agonist with Protein F binding molecules, recombinant Protein F binding molecules, natural substrates or ligands, or substrate or ligand mimetics, under appropriate conditions for a competitive inhibition assay. Protein F can be labeled, such as by radioactivity or a colorimetric compound, such that the number of Protein F molecules bound to a binding molecule or converted to product can be determined accurately to assess the effectiveness of the potential antagonist.

Potential antagonists include, among others, small organic molecules, peptides, polypeptides and antibodies that bind to a polynucleotide and/or polypeptide of the invention and thereby inhibit or extinguish its activity or expression. Potential antagonists also may be small organic molecules, a peptide, a polypeptide such as a closely related protein or antibody that binds the same sites on a binding molecule, such as a binding molecule, without inducing Protein F induced activities, thereby preventing the action or expression of Protein F polypeptides and/or polynucleotides by excluding Protein F polypeptides and/or polynucleotides from binding.

Potential antagonists include a small molecule that binds to and occupies the binding site of the polypeptide thereby preventing binding to cellular binding molecules, such that normal biological activity is prevented. Examples of small molecules include but are not limited to small organic molecules, peptides or peptide-like molecules. Other potential antagonists include antisense molecules (see Okano, J. Neurochem. 56: 560 (1991); OLIGODEOXYNUCLEOTIDES AS ANTISENSE INHIBITORS OF GENE EXPRESSION, CRC Press, Boca Raton, Fla. (1988), for a description of these molecules). Preferred potential antagonists include compounds related to and variants of Protein F.

In a further aspect, the present invention relates to genetically engineered soluble fusion proteins comprising a polypeptide of the present invention, or a fragment thereof, and various portions of the constant regions of heavy or light chains of immunoglobulins of various subclasses (IgG, IgM, IgA, IgE). Preferred as an immunoglobulin is the constant part of the heavy chain of human IgG, particularly IgG1, where fusion takes place at the hinge region. In a particular embodiment, the Fc part can be removed simply by incorporation of a cleavage sequence which can be cleaved with blood clotting factor Xa. Furthermore, this invention relates to processes for the preparation of these fusion proteins by genetic engineering, and to the use thereof for drug screening, diagnosis and therapy. A further aspect of the invention also relates to polynucleotides encoding such fusion proteins. Examples of fusion protein technology can be found in International Patent Application Nos. WO94/29458 and WO94/22914.

Each of the polynucleotide sequences provided herein may be used in the discovery and development of antibacterial compounds. The encoded protein, upon expression, can be used as a target for the screening of antibacterial drugs. Additionally, the polynucleotide sequences encoding the amino terminal regions of the encoded protein or Shine-Delgarno or other translation facilitating sequences of the respective mRNA can be used to construct antisense sequences to control the expression of the coding sequence of interest.

The invention also provides the use of the polypeptide, polynucleotide, agonist or antagonist of the invention to interfere with the initial physical interaction between a pathogen or pathogens and a eukaryotic, preferably mammalian, host responsible for sequalae of infection. In particular, the molecules of the invention may be used: in the prevention of adhesion of bacteria, in particular gram positive and/or gram negative bacteria, to eukaryotic, preferably mammalian, extracellular matrix proteins on in-dwelling devices or to extracellular matrix proteins in wounds; to block bacterial adhesion between eukaryotic, preferably mammalian, extracellular matrix proteins and bacterial Protein F proteins that mediate tissue damage and/or; to block the normal progression of pathogenesis in infections initiated other than by the implantation of in-dwelling devices or by other surgical techniques.

In accordance with yet another aspect of the invention, there are provided Protein F agonists and antagonists, preferably bacteristatic or bactericidal agonists and antagonists.

The antagonists and agonists of the invention may be employed, for instance, to prevent, inhibit and/or treat diseases.

In a further aspect, the present invention relates to mimotopes of the polypeptide of the invention. A mimotope is a peptide sequence, sufficiently similar to the native peptide (sequentially or structurally), which is capable of being recognised by antibodies which recognise the native peptide; or is capable of raising antibodies which recognise the native peptide when coupled to a suitable carrier.

Peptide mimotopes may be designed for a particular purpose by addition, deletion or substitution of elected amino acids. Thus, the peptides may be modified for the purposes of ease of conjugation to a protein carrier. For example, it may be desirable for some chemical conjugation methods to include a terminal cysteine. In addition it may be desirable for peptides conjugated to a protein carrier to include a hydrophobic terminus distal from the conjugated terminus of the peptide, such that the free unconjugated end of the peptide remains associated with the surface of the carrier protein. Thereby presenting the peptide in a conformation which most closely resembles that of the peptide as found in the context of the whole native molecule. For example, the peptides may be altered to have an N-terminal cysteine and a C-terminal hydrophobic amidated tail. Alternatively, the addition or substitution of a D-stereoisomer form of one or more of the amino acids (inverso sequences) may be performed to create a beneficial derivative, for example to enhance stability of the peptide. Mimotopes may also be retro sequences of the natural peptide sequences, in that the sequence orientation is reversed. Mimotopes may also be retro-inverso in character. Retro, inverso and retro-inverso peptides are described in WO 95/24916 and WO 94/05311.

Alternatively, peptide mimotopes may be identified using antibodies which are capable themselves of binding to the polypeptides of the present invention using techniques such as phage display technology (EP 0 552 267 B1). This technique generates a large number of peptide sequences which mimic the structure of the native peptides and are, therefore, capable of binding to anti-native peptide antibodies, but may not necessarily themselves share significant sequence homology to the native polypeptide.

### Vaccines

Another aspect of the invention relates to a method for inducing an immunological response in an individual, particularly a mammal, preferably humans, which comprises inoculating the individual with Protein F polynucleotide and/or polypeptide, or a fragment or variant thereof, adequate to produce antibody and/or T cell immune response to protect said individual from infection, particularly bacterial infection and most particularly non typeable H. influenzae infection. Also provided are methods whereby such immunological response slows bacterial replication. Yet another aspect of the invention relates to a method of inducing immunological response in an individual which comprises delivering to such individual a nucleic acid vector, sequence or ribozyme to direct expression of Protein F polynucleotides and/or polypeptides, or a fragment or a variant thereof, for expressing Protein F polynucleotides and/or polypeptides, or a fragment or a variant thereof in vivo in order to induce an immunological response, such as, to produce antibody and/or T cell immune response, including, for example, cytokine-producing T cells or cytotoxic T cells, to protect said individual, preferably a human, from disease, whether that disease is already established within the individual or not. One example of administering the gene is by accelerating it into the desired cells as a coating on particles or otherwise. Such nucleic acid vector may comprise DNA, RNA, a ribozyme, a modified nucleic acid, a DNA/RNA hybrid, a DNA-protein complex or an RNA-protein complex.

A further aspect of the invention relates to an immunological composition that when introduced into an individual, preferably a human, capable of having induced within it an immunological response, induces an immunological response in such individual to a Protein F polynucleotide and/or polypeptide encoded therefrom, wherein the composition comprises a recombinant Protein F polynucleotide and/or polypeptide encoded therefrom and/or comprises DNA and/or RNA which encodes and expresses an antigen of said Protein F polynucleotide, polypeptide encoded therefrom, or other polypeptide of the invention. The immunological response may be used therapeutically or prophylactically and may take the form of antibody immunity and/or cellular immunity, such as cellular immunity arising from CTL or CD4+ T cells.

Protein F polypeptides or a fragment thereof may be fused with co-protein or chemical moiety which may or may not by itself produce antibodies, but which is capable of stabilizing the first protein and producing a fused or modified protein which will have antigenic and/or immunogenic properties, and preferably protective properties. Thus fused recombinant protein, preferably further comprises an antigenic co-protein, such as lipoprotein or protein D from Haemophilus influenzae (EP 594610), protein E from H. influenzae (EP 1 973 933), Glutathione-S-transferase (GST) or beta-galactosidase, or any other relatively large co-protein which solubilizes the protein and facilitates production and purification thereof. Moreover, the co-protein may act as an adjuvant in the sense of providing a generalized stimulation of the immune system of the organism receiving the protein. The co-protein may be attached to either the amino- or carboxy-terminus of the first protein.

In a vaccine composition according to the invention, Protein F polypeptides and/or polynucleotides, or a fragment, or a mimotope, or a variant thereof may be present in a vector, such as the live recombinant vectors described above for example live bacterial vectors.

Also suitable are non-live vectors for the Protein F polypeptides, for example bacterial outer-membrane vesicles or “blebs”. OM blebs are derived from the outer membrane of the two-layer membrane of Gram-negative bacteria and have been documented in many Gram-negative bacteria (Zhou, L et al. 1998. FEMS Microbiol. Lett. 163:223-228) including C. trachomatis and C. psittaci. A non-exhaustive list of bacterial pathogens reported to produce blebs also includes: Bordetella pertussis, Borrelia burgdorferi, Brucella melitensis, Brucella ovis, Esherichia coli, Haemophilus influenzae, Legionella pneumophila, Moraxella catarrhalis, Neisseria gonorrhoeae, Neisseria meningitidis, Pseudomonas aeruginosa and Yersinia enterocolitica.

Blebs have the advantage of providing outer-membrane proteins in their native conformation and are thus particularly useful for vaccines. Blebs can also be improved for vaccine use by engineering the bacterium so as to modify the expression of one or more molecules at the outer membrane. Thus for example the expression of a desired immunogenic protein at the outer membrane, such as the Protein F polypeptides, can be introduced or upregulated (e.g. by altering the promoter). Instead or in addition, the expression of outer-membrane molecules which are either not relevant (e.g. unprotective antigens or immunodominant but variable proteins) or detrimental (e.g. toxic molecules such as LPS, or potential inducers of an autoimmune response) can be downregulated. These approaches are discussed in more detail below.

The non-coding flanking regions of the Protein F genes contain regulatory elements important in the expression of the gene. This regulation takes place both at the transcriptional and translational level. The sequence of these regions, either upstream or downstream of the open reading frame of the gene, can be obtained by DNA sequencing. This sequence information allows the determination of potential regulatory motifs such as the different promoter elements, terminator sequences, inducible sequence elements, repressors, elements responsible for phase variation, the shine-dalgarno sequence, regions with potential secondary structure involved in regulation, as well as other types of regulatory motifs or sequences. This sequence is a further aspect of the invention.

This sequence information allows the modulation of the natural expression of the Protein F genes. The upregulation of the gene expression may be accomplished by altering the promoter, the shine-dalgarno sequence, potential repressor or operator elements, or any other elements involved. Likewise, downregulation of expression can be achieved by similar types of modification. Alternatively, by changing phase variation sequences, the expression of the gene can be put under phase variation control, or it may be uncoupled from this regulation. In another approach, the expression of the gene can be put under the control of one or more inducible elements allowing regulated expression. Examples of such regulation include, but are not limited to, induction by temperature shift, addition of inductor substrates like selected carbohydrates or their derivatives, trace elements, vitamins, co-factors, metal ions, etc.

Such modifications as described above can be introduced by several different means. The modification of sequences involved in gene expression can be carried out in vivo by random mutagenesis followed by selection for the desired phenotype. Another approach consists in isolating the region of interest and modifying it by random mutagenesis, or site-directed replacement, insertion or deletion mutagenesis. The modified region can then be reintroduced into the bacterial genome by homologous recombination, and the effect on gene expression can be assessed. In another approach, the sequence knowledge of the region of interest can be used to replace or delete all or part of the natural regulatory sequences. In this case, the regulatory region targeted is isolated and modified so as to contain the regulatory elements from another gene, a combination of regulatory elements from different genes, a synthetic regulatory region, or any other regulatory region, or to delete selected parts of the wild-type regulatory sequences. These modified sequences can then be reintroduced into the bacterium via homologous recombination into the genome. A non-exhaustive list of preferred promoters that could be used for upregulation of gene expression includes the promoters porA, porB, IbpB, tbpB, p110, lst, hpuAB from N. meningitidis or N. gonorroheae; ompCD, copB, IbpB, ompE, UspA1; UspA2; TbpB from M. catarrhalis; p1, p2, p4, p5, p6, IpD, pE, tbpB, D15, Hia, Hmw1, Hmw2 from H. influenzae.

In one example, the expression of the gene can be modulated by exchanging its promoter with a stronger promoter (through isolating the upstream sequence of the gene, in vitro modification of this sequence, and reintroduction into the genome by homologous recombination). Upregulated expression can be obtained in both the bacterium as well as in the outer membrane vesicles shed (or made) from the bacterium.

In other examples, the described approaches can be used to generate recombinant bacterial strains with improved characteristics for vaccine applications. These can be, but are not limited to, attenuated strains, strains with increased expression of selected antigens, strains with knock-outs (or decreased expression) of genes interfering with the immune response, strains with modulated expression of immunodominant proteins, strains with modulated shedding of outer-membrane vesicles.

Thus, also provided by the invention is a modified upstream region of the Protein F genes, which modified upstream region contains a heterologous regulatory element which alters the expression level of the Protein F proteins located at the outer membrane. The upstream region according to this aspect of the invention includes the sequence upstream of the Protein F genes. The upstream region starts immediately upstream of the Protein F genes and continues usually to a position no more than about 1000 bp upstream of the gene from the ATG start codon. In the case of a gene located in a polycistronic sequence (operon) the upstream region can start immediately preceding the gene of interest, or preceding the first gene in the operon. Preferably, a modified upstream region according to this aspect of the invention contains a heterologous promotor at a position between 500 and 700 bp upstream of the ATG.

The use of the disclosed upstream regions to upregulate the expression of the Protein F genes, a process for achieving this through homologous recombination (for instance as described in WO 01/09350 incorporated by reference herein), a vector comprising upstream sequence suitable for this purpose, and a host cell so altered are all further aspects of this invention.

Thus, the invention provides Protein F polypeptides, in a modified bacterial bleb. The invention further provides modified host cells capable of producing the non-live membrane-based bleb vectors. The invention further provides nucleic acid vectors comprising the Protein F genes having a modified upstream region containing a heterologous regulatory element.

Further provided by the invention are processes to prepare the host cells and bacterial blebs according to the invention.

Also provided by this invention are compositions, particularly vaccine compositions, and methods comprising the polypeptides and/or polynucleotides of the invention and immunostimulatory DNA sequences, such as those described in Sato, Y. et al. Science 273: 352 (1996).

Also, provided by this invention are methods using the described polynucleotide or particular fragments thereof, which have been shown to encode non-variable regions of bacterial cell surface proteins, in polynucleotide constructs used in such genetic immunization experiments in animal models of infection with H. influenzae. Such experiments will be particularly useful for identifying protein epitopes able to provoke a prophylactic or therapeutic immune response. It is believed that this approach will allow for the subsequent preparation of monoclonal antibodies of particular value, derived from the requisite organ of the animal successfully resisting or clearing infection, for the development of prophylactic agents or therapeutic treatments of bacterial infection, particularly H. influenzae infection, in mammals, particularly humans.

The invention also includes a vaccine formulation which comprises an immunogenic recombinant polypeptide and/or polynucleotide of the invention together with a suitable carrier, such as a pharmaceutically acceptable carrier. Since the polypeptides and polynucleotides may be broken down in the stomach, each is preferably administered parenterally, including, for example, administration that is subcutaneous, intramuscular, intravenous, or intradermal. Formulations suitable for parenteral administration include aqueous and non-aqueous sterile injection solutions which may contain anti-oxidants, buffers, bacteriostatic compounds and solutes which render the formulation isotonic with the bodily fluid, preferably the blood, of the individual; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use.

The vaccine formulation of the invention may also include adjuvant systems for enhancing the immunogenicity of the formulation. Preferably the adjuvant system raises preferentially a TH1 type of response.

An immune response may be broadly distinguished into two extreme categories, being a humoral or cell mediated immune responses (traditionally characterised by antibody and cellular effector mechanisms of protection respectively). These categories of response have been termed TH1-type responses (cell-mediated response), and TH2-type immune responses (humoral response).

Extreme TH1-type immune responses may be characterised by the generation of antigen specific, haplotype restricted cytotoxic T lymphocytes, and natural killer cell responses. In mice TH1-type responses are often characterised by the generation of antibodies of the IgG2a subtype, whilst in the human these correspond to IgG1 type antibodies. TH2-type immune responses are characterised by the generation of a broad range of immunoglobulin isotypes including in mice IgG1, IgA, and IgM.

It can be considered that the driving force behind the development of these two types of immune responses are cytokines. High levels of TH1-type cytokines tend to favour the induction of cell mediated immune responses to the given antigen, whilst high levels of TH2-type cytokines tend to favour the induction of humoral immune responses to the antigen.

The distinction of TH1 and TH2-type immune responses is not absolute. In reality an individual will support an immune response which is described as being predominantly TH1 or predominantly TH2. However, it is often convenient to consider the families of cytokines in terms of that described in murine CD4 +ve T cell clones by Mosmann and Coffman (Mosmann, T. R. and Coffman, R. L. (1989) TH1 and TH2 cells: different patterns of lymphokine secretion lead to different functional properties. Annual Review of Immunology, 7, p 145-173). Traditionally, TH1-type responses are associated with the production of the INF-γ and IL-2 cytokines by T-lymphocytes. Other cytokines often directly associated with the induction of TH1-type immune responses are not produced by T-cells, such as IL-12. In contrast, TH2-type responses are associated with the secretion of IL-4, IL-5, IL-6 and IL-13.

It is known that certain vaccine adjuvants are particularly suited to the stimulation of either TH1 or TH2-type cytokine responses. Traditionally the best indicators of the TH1:TH2 balance of the immune response after a vaccination or infection includes direct measurement of the production of TH1 or TH2 cytokines by T lymphocytes in vitro after restimulation with antigen, and/or the measurement of the IgG1:IgG2a ratio of antigen specific antibody responses.

Thus, a TH1-type adjuvant is one which preferentially stimulates isolated T-cell populations to produce high levels of TH1-type cytokines when re-stimulated with antigen in vitro, and promotes development of both CD8+ cytotoxic T lymphocytes and antigen specific immunoglobulin responses associated with TH1-type isotype.

Adjuvants which are capable of preferential stimulation of the TH1 cell response are described in International Patent Application No. WO 94/00153 and WO 95/17209.

3 De-O-acylated monophosphoryl lipid A (3D-MPL) is one such adjuvant. This is known from GB 2220211 (Ribi). Chemically it is a mixture of 3 De-O-acylated monophosphoryl lipid A with 4, 5 or 6 acylated chains and is manufactured by Ribi Immunochem, Montana. A preferred form of 3 De-O-acylated monophosphoryl lipid A is disclosed in European Patent 0 689 454 B1 (SmithKline Beecham Biologicals SA).

Preferably, the particles of 3D-MPL are small enough to be sterile filtered through a 0.22 micron membrane (European Patent number 0 689 454).

3D-MPL will be present in the range of 10 μg-100 μg preferably 25-50 μg per dose wherein the antigen will typically be present in a range 2-50 μg per dose.

Another preferred adjuvant comprises QS21, an Hplc purified non-toxic fraction derived from the bark of Quillaja Saponaria Molina. Optionally this may be admixed with 3 De-O-acylated monophosphoryl lipid A (3D-MPL), optionally together with a carrier.

The method of production of QS21 is disclosed in U.S. Pat. No. 5,057,540.

Non-reactogenic adjuvant formulations containing QS21 have been described previously (WO 96/33739). Such formulations comprising QS21 and cholesterol have been shown to be successful TH1 stimulating adjuvants when formulated together with an antigen.

Further adjuvants which are preferential stimulators of TH1 cell response include immunomodulatory oligonucleotides, for example unmethylated CpG sequences as disclosed in WO 96/02555.

Combinations of different TH1 stimulating adjuvants, such as those mentioned hereinabove, are also contemplated as providing an adjuvant which is a preferential stimulator of TH1 cell response. For example, QS21 can be formulated together with 3D-MPL. The ratio of QS21:3D-MPL will typically be in the order of 1:10 to 10:1; preferably 1:5 to 5:1 and often substantially 1:1. The preferred range for optimal synergy is 2.5:1 to 1:1 3D-MPL: QS21.

Preferably a carrier is also present in the vaccine composition according to the invention. The carrier may be an oil in water emulsion, or an aluminium salt, such as aluminium phosphate or aluminium hydroxide.

A preferred oil-in-water emulsion comprises a metabolisible oil, such as squalene, alpha tocopherol and Tween 80. In a particularly preferred aspect the antigens in the vaccine composition according to the invention are combined with QS21 and 3D-MPL in such an emulsion. Additionally the oil in water emulsion may contain span 85 and/or lecithin and/or tricaprylin.

Typically for human administration QS21 and 3D-MPL will be present in a vaccine in the range of 1 μg-200 μg, such as 10-100 μg, preferably 10 μg-50 μg per dose. Typically the oil in water will comprise from 2 to 10% squalene, from 2 to 10% alpha tocopherol and from 0.3 to 3% tween 80. Preferably the ratio of squalene:alpha tocopherol is equal to or less than 1 as this provides a more stable emulsion. Span 85 may also be present at a level of 1%. In some cases it may be advantageous that the vaccines of the present invention will further contain a stabiliser.

Non-toxic oil in water emulsions preferably contain a non-toxic oil, e.g. squalane or squalene, an emulsifier, e.g. Tween 80, in an aqueous carrier. The aqueous carrier may be, for example, phosphate buffered saline.

A particularly potent adjuvant formulation involving QS21, 3D-MPL and tocopherol in an oil in water emulsion is described in WO 95/17210.

While the invention has been described with reference to certain Protein F polypeptides and polynucleotides, it is to be understood that this covers fragments of the naturally occurring polypeptides and polynucleotides, and similar polypeptides and polynucleotides with additions, deletions or substitutions which do not substantially affect the immunogenic properties of the recombinant polypeptides or polynucleotides. Preferred fragments/peptides are shown in FIG. 11.

The present invention also provides a polyvalent vaccine composition comprising a vaccine formulation of the invention in combination with other antigens, in particular antigens useful for treating otitis media. Such a polyvalent vaccine composition may include a TH-1 inducing adjuvant as hereinbefore described.

In a preferred embodiment, the polypeptides, fragments and immunogens of the invention are formulated with one or more of the following groups of antigens: a) one or more pneumococcal capsular polysaccharides (either plain or conjugated to a carrier protein); b) one or more antigens that can protect a host against M. catarrhalis infection; c) one or more protein antigens that can protect a host against Streptococcus pneumoniae infection; d) one or more further non typeable Haemophilus influenzae protein antigens; e) one or more antigens that can protect a host against RSV; and f) one or more antigens that can protect a host against influenza virus. Combinations with: groups a) and b); b) and c); b), d), and a) and/or c); b), d), e), f), and a) and/or c) are preferred. Such vaccines may be advantageously used as global otitis media vaccines.

The pneumococcal capsular polysaccharide antigens are preferably selected from serotypes 1, 2, 3, 4, 5, 6B, 7F, 8, 9N, 9V, 10A, 11A, 12F, 14, 15B, 17F, 18C, 19A, 19F, 20, 22F, 23F and 33F (most preferably from serotypes 1, 3, 4, 5, 6B, 7F, 9V, 14, 18C, 19F and 23F).

Preferred pneumococcal protein antigens are those pneumococcal proteins which are exposed on the outer surface of the pneumococcus (capable of being recognised by a host's immune system during at least part of the life cycle of the pneumococcus), or are proteins which are secreted or released by the pneumococcus. Most preferably, the protein is a toxin, adhesin, 2-component signal tranducer, or lipoprotein of Streptococcus pneumoniae, or fragments thereof. Particularly preferred proteins include, but are not limited to: pneumolysin (preferably detoxified by chemical treatment or mutation) [Mitchell et al. Nucleic Acids Res. 1990 Jul. 11; 18(13): 4010 “Comparison of pneumolysin genes and proteins from Streptococcus pneumoniae types 1 and 2.”, Mitchell et al. Biochim Biophys Acta 1989 January 23; 1007(1): 67-72 “Expression of the pneumolysin gene in Escherichia coli: rapid purification and biological properties.”, WO 96/05859 (A. Cyanamid), WO 90/06951 (Paton et al), WO 99/03884 (NAVA)]; PspA and transmembrane deletion variants thereof (WO 92/14488; WO 99/53940; U.S. Pat. No. 5,804,193—Briles et al.); PspC and transmembrane deletion variants thereof (WO 99/53940; WO 97/09994—Briles et al); PsaA and transmembrane deletion variants thereof (Berry & Paton, Infect Immun 1996 December; 64(12):5255-62 “Sequence heterogeneity of PsaA, a 37-kilodalton putative adhesin essential for virulence of Streptococcus pneumoniae”); pneumococcal choline binding proteins and transmembrane deletion variants thereof; CbpA and transmembrane deletion variants thereof (WO 97/41151; WO 99/51266); Glyceraldehyde-3-phosphate-dehydrogenase (Infect. Immun. 1996 64:3544); HSP70 (WO 96/40928); PcpA (Sanchez-Beato et al. FEMS Microbiol Lett 1998, 164:207-14); M like protein, SB patent application No. EP 0837130; and adhesin 18627 (SB Patent application No. EP 0834568). Further preferred pneumococcal protein antigens are those disclosed in WO 98/18931, particularly those selected in WO 98/18930 and PCT/US99/30390.

Preferred Moraxella catarrhalis protein antigens which can be included in a combination vaccine (especially for the prevention of otitis media) are: OMP106 [WO 97/41731 (Antex) & WO 96/34960 (PMC)]; OMP21; LbpA &/or LbpB [WO 98/55606 (PMC)]; TbpA &/or TbpB [WO 97/13785 & WO 97/32980 (PMC)]; CopB [Helminen M E, et al. (1993) Infect. Immun. 61:2003-2010]; UspA1 and/or UspA2 [WO 2007/018463 (Arne Forsgren AB), WO 93/03761 (University of Texas)]; OmpCD; HasR (PCT/EP99/03824); PiIQ (PCT/EP99/03823); OMP85 (PCT/EP00/01468); lipo06 (GB 9917977.2); lipo10 (GB 9918208.1); lipo11 (GB 9918302.2); lipo18 (GB 9918038.2); P6 (PCT/EP99/03038); D15 (PCT/EP99/03822); OmpIA1 (PCT/EP99/06781); Hly3 (PCT/EP99/03257); and OmpE.

Preferred further non-typeable Haemophilus influenzae protein antigens which can be included in a combination vaccine (especially for the prevention of otitis media) include: Fimbrin protein [(U.S. Pat. No. 5,766,608—Ohio State Research Foundation)] and fusions comprising peptides therefrom [eg LB1(f) peptide fusions; U.S. Pat. No. 5,843,464 (OSU) or WO 99/64067]; OMP26 [WO 97/01638 (Cortecs)]; P6 [EP 281673 (State University of New York)]; protein D (EP 594610); protein E (EP 1 973 933); TbpA and/or TbpB; Hia; Hsf; Hin47; Hit Hmw1; Hmw2; Hmw3; Hmw4; Hap; D15 (WO 94/12641); P2; P5 (WO 94/26304); NIpC2 (BASB205) [WO 02/30971]; Slp (BASB203) [WO 02/30960]; and iOMP1681 (BASB210) [WO 02/34772].

Preferred influenza virus antigens include whole, live or inactivated virus, split influenza virus, grown in eggs or MDCK cells, or Vero cells or whole flu virosomes (as described by R. Gluck, Vaccine, 1992, 10, 915-920) or purified or recombinant proteins thereof, such as HA, NP, NA, or M proteins, or combinations thereof.

Preferred RSV (Respiratory Syncytial Virus) antigens include the F glycoprotein, the G glycoprotein, the HN protein, or derivatives thereof.

### Compositions, Kits and Administration

In a further aspect of the invention there are provided compositions comprising Protein F polynucleotides and/or Protein F polypeptides for administration to a cell or to a multicellular organism.

The invention also relates to compositions comprising a polynucleotide and/or a polypeptide discussed herein or their agonists or antagonists. The polypeptides and polynucleotides of the invention may be employed in combination with a non-sterile or sterile carrier or carriers for use with cells, tissues or organisms, such as a pharmaceutical carrier suitable for administration to an individual. Such compositions comprise, for instance, a media additive or a therapeutically effective amount of a polypeptide and/or polynucleotide of the invention and a pharmaceutically acceptable carrier or excipient. Such carriers may include, but are not limited to, saline, buffered saline, dextrose, water, glycerol, ethanol and combinations thereof. The formulation should suit the mode of administration. The invention further relates to diagnostic and pharmaceutical packs and kits comprising one or more containers filled with one or more of the ingredients of the aforementioned compositions of the invention.

Polypeptides, polynucleotides and other compounds of the invention may be employed alone or in conjunction with other compounds, such as therapeutic compounds.

The pharmaceutical compositions may be administered in any effective, convenient manner including, for instance, administration by topical, oral, anal, vaginal, intravenous, intraperitoneal, intramuscular, subcutaneous, intranasal or intradermal routes among others.

In therapy or as a prophylactic, the active agent may be administered to an individual as an injectable composition, for example as a sterile aqueous dispersion, preferably isotonic.

In a further aspect, the present invention provides for pharmaceutical compositions comprising a therapeutically effective amount of a polypeptide and/or polynucleotide, such as the soluble form of a polypeptide and/or polynucleotide of the present invention, agonist or antagonist peptide or small molecule compound, in combination with a pharmaceutically acceptable carrier or excipient. Such carriers include, but are not limited to, saline, buffered saline, dextrose, water, glycerol, ethanol, and combinations thereof. The invention further relates to pharmaceutical packs and kits comprising one or more containers filled with one or more of the ingredients of the aforementioned compositions of the invention. Polypeptides, polynucleotides and other compounds of the present invention may be employed alone or in conjunction with other compounds, such as therapeutic compounds.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if a polypeptide or other compounds of the present invention can be formulated in an enteric or an encapsulated formulation, oral administration may also be possible. Administration of these compounds may also be topical and/or localized, in the form of salves, pastes, gels, solutions, powders and the like.

For administration to mammals, and particularly humans, it is expected that the daily dosage level of the active agent will be from 0.01 mg/kg to 10 mg/kg, typically around 1 mg/kg. The physician in any event will determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention.

The dosage range required depends on the choice of peptide, the route of administration, the nature of the formulation, the nature of the subject's condition, and the judgment of the attending practitioner. Suitable dosages, however, are in the range of 0.1-100 pg/kg of subject.

A vaccine composition is conveniently in injectable form. Conventional adjuvants may be employed to enhance the immune response. A suitable unit dose for vaccination is 0.5-5 microgram/kg of antigen, and such dose is preferably administered 1-3 times and with an interval of 1-3 weeks. With the indicated dose range, no adverse toxicological effects will be observed with the compounds of the invention which would preclude their administration to suitable individuals.

Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

### Sequence Databases, Sequences in a Tangible Medium, and Algorithms

Polynucleotide and polypeptide sequences form a valuable information resource with which to determine their 2- and 3-dimensional structures as well as to identify further sequences of similar homology. These approaches are most easily facilitated by storing the sequence in a computer readable medium and then using the stored data in a known macromolecular structure program or to search a sequence database using well known searching tools, such as the GCG program package.

Also provided by the invention are methods for the analysis of character sequences or strings, particularly genetic sequences or encoded protein sequences. Preferred methods of sequence analysis include, for example, methods of sequence homology analysis, such as identity and similarity analysis, DNA, RNA and protein structure analysis, sequence assembly, cladistic analysis, sequence motif analysis, open reading frame determination, nucleic acid base calling, codon usage analysis, nucleic acid base trimming, and sequencing chromatogram peak analysis.

A computer based method is provided for performing homology identification. This method comprises the steps of: providing a first polynucleotide sequence comprising the sequence of a polynucleotide of the invention in a computer readable medium; and comparing said first polynucleotide sequence to at least one second polynucleotide or polypeptide sequence to identify homology.

A computer based method is also provided for performing homology identification, said method comprising the steps of: providing a first polypeptide sequence comprising the sequence of a polypeptide of the invention in a computer readable medium; and comparing said first polypeptide sequence to at least one second polynucleotide or polypeptide sequence to identify homology.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### DEFINITIONS

“Identity,” as known in the art, is a relationship between two or more polypeptide sequences or two or more polynucleotide sequences, as the case may be, as determined by comparing the sequences. In the art, “identity” also means the degree of sequence relatedness between polypeptide or polynucleotide sequences, as the case may be, as determined by the match between strings of such sequences. “Identity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GAP program in the GCG program package (Devereux, J., et al., Nucleic Acids Research 12(1): 387 (1984)), BLASTP, BLASTN (Altschul, S. F. et al., J. Molec. Biol. 215: 403-410 (1990), and FASTA (Pearson and Lipman Proc. Natl. Acad. Sci. USA 85; 2444-2448 (1988). The BLAST family of programs is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Parameters for polypeptide sequence comparison include the following:

### Algorithm: Needleman and Wunsch, J. Mol Biol. 48: 443-453 (1970)

Comparison matrix: BLOSSUM62 from Henikoff and Henikoff, Proc. Natl. Acad. Sci. USA. 89:10915-10919 (1992)

### Gap Penalty: 8 Gap Length Penalty: 2

A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following:

### Algorithm: Needleman and Wunsch, J. Mol Biol. 48: 443-453 (1970)

Comparison matrix: matches=+10, mismatch=0

### Gap Penalty: 50

### Gap Length Penalty: 3

Available as: The “gap” program from Genetics Computer Group, Madison Wis. These are the default parameters for nucleic acid comparisons.

A preferred meaning for “identity” for polynucleotides and polypeptides, as the case may be, are provided in (1) and (2) below. (1) Polynucleotide embodiments further include an isolated polynucleotide comprising a polynucleotide sequence having at least a 50, 60, 70, 80, 85, 90, 95, 97 or 100% identity to the reference sequence of SEQ ID NO: 15, wherein said polynucleotide sequence may be identical to the reference sequence of SEQ ID NO: 15 or may include up to a certain integer number of nucleotide alterations as compared to the reference sequence, wherein said alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein said number of nucleotide alterations is determined by multiplying the total number of nucleotides in SEQ ID NO: 15 by the integer defining the percent identity divided by 100 and then subtracting that product from said total number of nucleotides in SEQ ID NO: 15, or:

nₙ≦xₙ−(xₙ·y),

wherein nₙ is the number of nucleotide alterations, xₙ is the total number of nucleotides in SEQ ID NO: 15, y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and · is the symbol for the multiplication operator, and wherein any non-integer product of xₙ and y is rounded down to the nearest integer prior to subtracting it from xₙ. Alterations of polynucleotide sequences encoding the polypeptides of SEQ ID NO:1-14 may create nonsense, missense or frameshift mutations in this coding sequence and thereby alter the polypeptide encoded by the polynucleotide following such alterations.

By way of example, a polynucleotide sequence of the present invention may be identical to the reference sequences of SEQ ID NO: 15, that is it may be 100% identical, or it may include up to a certain integer number of nucleic acid alterations as compared to the reference sequence such that the percent identity is less than 100% identity. Such alterations are selected from the group consisting of at least one nucleic acid deletion, substitution, including transition and transversion, or insertion, and wherein said alterations may occur at the 5′ or 3′ terminal positions of the reference polynucleotide sequence or anywhere between those terminal positions, interspersed either individually among the nucleic acids in the reference sequence or in one or more contiguous groups within the reference sequence. The number of nucleic acid alterations for a given percent identity is determined by multiplying the total number of nucleic acids in SEQ ID NO: 15 by the integer defining the percent identity divided by 100 and then subtracting that product from said total number of nucleic acids in SEQ ID NO: 11, or:

nₙ≦xₙ−(xₙ·y),

wherein nₙ is the number of nucleic acid alterations, xₙ is the total number of nucleic acids in SEQ ID NO: 15, y is, for instance 0.70 for 70%, 0.80 for 80%, 0.85 for 85% etc., · is the symbol for the multiplication operator, and wherein any non-integer product of xₙ and y is rounded down to the nearest integer prior to subtracting it from xₙ.

(2) Polypeptide embodiments further include an isolated polypeptide comprising a polypeptide having at least a 50,60, 70, 80, 85, 90, 95, 97 or 100% identity to the polypeptide reference sequence of SEQ ID NO:1-14, wherein said polypeptide sequence may be identical to the reference sequence of SEQ ID NO:1-14 or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids in SEQ ID NO:1-14 by the integer defining the percent identity divided by 100 and then subtracting that product from said total number of amino acids in SEQ ID NO:1-14, respectively, or:

nₐ≦xₐ−(xₐ·y),

wherein nₐ is the number of amino acid alterations, xₐ is the total number of amino acids in SEQ ID NO:1-14, y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and · is the symbol for the multiplication operator, and wherein any non-integer product of xₐ and y is rounded down to the nearest integer prior to subtracting it from xₐ.

By way of example, a polypeptide sequence of the present invention may be identical to the reference sequence of SEQ ID NO:1-14, that is it may be 100% identical, or it may include up to a certain integer number of amino acid alterations as compared to the reference sequence such that the percent identity is less than 100% identity. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:1-14 by the integer defining the percent identity divided by 100 and then subtracting that product from said total number of amino acids in SEQ ID NO:1-14, or:

nₐ≦xₐ−(xₐ·y),

wherein nₐ is the number of amino acid alterations, xₐ is the total number of amino acids in SEQ ID NO: 1-14, y is, for instance 0.70 for 70%, 0.80 for 80%, 0.85 for 85% etc., and · is the symbol for the multiplication operator, and wherein any non-integer product of xₐ and y is rounded down to the nearest integer prior to subtracting it from xₐ.

“Individual(s),” when used herein with reference to an organism, means a multicellular eukaryote, including, but not limited to a metazoan, a mammal, an ovid, a bovid, a simian, a primate, and a human.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Polynucleotide(s)” generally refers to any polyribonucleotide or polydeoxyribonucleotide, which may be unmodified RNA or DNA or modified RNA or DNA including single and double-stranded regions.

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Disease(s)” means any disease caused by or related to infection by a bacteria, including, for example, otitis media in infants and children, pneumonia in elderlies, sinusitis, nosocomial infections and invasive diseases, chronic otitis media with hearing loss, fluid accumulation in the middle ear, auditive nerve damage, delayed speech learning, infection of the upper respiratory tract and inflammation of the middle ear.

### Experimental Part

The examples below are carried out using standard techniques, which are well known and routine to those of skill in the art, except where otherwise described in detail. The examples are illustrative, but do not limit the invention. The present investigation describes the isolation, purification, characterization, cloning and expression of the novel vitronectin-binding outer membrane protein named protein F (pF) of H. influenzae, which was discovered using vitronectin as a bate, and the novel truncated recombinant protein F (pF).

### Materials and Methods

### Bacteria, Reagents and Epithelial Cell Lines

The non-typable H. influenzae strain NTHi3655 was a clinical isolate and a kind gift from R. Munson (Ohio State University, Colombus, Ohio). Clinical NTHi isolates were obtained by nasopharyngeal swabs from patients (Skåne County, Sweden) with upper respiratory tract infections. H. influenzae were grown overnight in brain heart infusion (BHI) broth (Difco Laboratories, Detroit, Mich.) supplemented with NAD and hemin (Sigma, St. Louis, Mo.) or on chocolate blood agar plates as described (Ronander et al., 2009).

A549 (CCL-185), and H292 epithelial cell lines were from ATCC. Both cell lines were maintained in RPMI 1640 with 10% FCS at 37° C. and 5% CO₂.

### Two-Dimensional SDS-Polyacrylamide Gel Electrophoresis (2D-SDS-PAGE)

Outer membrane vesicles (OMV) and outer membrane proteins were purified as described (Ronander et al., 2008, Schaar et al., 2010). OMV were subjected to isoelectric focusing (IEF) using the IPGphor IEF System (Amersham Pharmacia Biotech) (Ronander et al., 2008). For gel calibration, a standard was used (cat. no. 161-0320; Bio-Rad). 2-D polyacrylamide gels were electroblotted to Immobilon-PVDF filters (0.45 mm; Millipore, Bedford, Mass.) at 120 mA overnight. Spots in 2D-SDS-PAGE stained with Coomassie blue were cut out from gels and sent for sequencing by MALDI-ToF as described (Schaar et al., 2010).

### SDS-PAGE and Detection of Proteins on Membranes (Western Blot; Immunoblot)

SDS-PAGE was run at 150 constant voltage using 10% Bis-Tris gels with reagents as well as a blotting instrument from Novex (San Diego, Calif.) (Vidakovics et al., 2010). Gels were stained with Coomassie Brilliant Blue R-250 (Bio-Rad, Sundbyberg, Sweden). After electrophoretical transfer, the Immobilon-P membrane was blocked in PBS with 0.05% Tween 20 (PBS-Tween) containing 5% milk powder. After washings, membranes were incubated with human vitronectin (Sigma) (0.5 pg/ml) in PBS-Tween including 2% milk powder at room temperature. In some experiments, HRP-conjugated mouse anti-human vitronectin diluted 1/1,000 was added after washings. After incubation, development was performed in a Fluor-S Max or with ECL Western blotting detection reagents (Amersham Pharmacia Biotech, Uppsala, Sweden).

DNA Cloning and Protein Expression of Recombinant pF 12-293 in E. coli

Chromosomal DNA from NTHi 3655 was used as template to isolate the pF coding sequence. Restriction enzyme sites BamHI and HindIII were introduced by PCR into the flanking regions of the DNA encoding pF12-293. To fuse 6 histidine residues encoded by the expression vector, the pF12-293 stop codon was mutated. The resulting PCR product was ligated into pET26(+) (Novagen, Darmstadt, Germany). Plasmids encoding pF were transformed into the expressing host BL21(DE3). To produce recombinant pF12-293, bacteria were induced with isopropyl β-D-1-thiogalactopyranoside (IPTG) for 3.5 h. After centrifugation, the bacterial pellet was incubated with 1 mg/ml of lysozyme on ice, sonicated and centrifugated. Soluble protein was purified under native conditions on columns containing a nickel resin as recommended by the manufacturer (Novagen).

### Antibodies and Enzyme-Linked Immosorbent Assay (ELISA)

To produce a specific anti-pF antiserum, rabbits were immunized three times (with 2 weeks interval) intramuscularly with recombinant pF12-293 according to standard procedures using complete and incomplete Freunds adjuvants (Ronander et al. 2008). Resulting polyclonal antibodies (pAb) were isolated by affinity chromatography using pF12-293 conjugated to CnBr-Sepharose. In addition, anti-pF44-68 pAb were isolated by affinity chromatography using the specific pF44-68 peptide conjugated to CnBr-Sepharose. Horseradish peroxidase (HRP)-conjugated swine anti-rabbit polyclonal immunoglobulins were from Dakopatts (Gentofte, Denmark). Recombinant truncated Moraxella catarrhalis (non-IgD binding) IgD binding protein (MID) 962-1200 was used as a negative control (Nordstrom et al., 2002).

Mice (Balb/c) were also immunized with recombinant pF12-293 as described (Ronander et al., 2009). Specific mouse anti-pF pAb were analysed by ELISA. Briefly, 50 μg recombinant pF12-293 was coated in microtiterplates overnight at 4° C. After a wash, mouse sera were added and incubated at room temperature. After 30 mins and additional washes, HRP-conjugated rabbit ant-mouse polyclonal antibodies were added and absorbance read at OD₅₄₀.

Manufacture of a pF-Deficient H. influenzae (NTHi 3655 Δpf)

Genomic DNA isolated from NTHi 3655 was used as template. The 5′- and 3″-ends of pf were amplified and fused with a cassette comprising the chloramphenicol acetyltransferase (cat) gene by using PCR by overlapping extension (Riesbeck et al., 1999). A specific uptake sequence (AAGTGCGGT) was included in one of the flanking primers. The resulting PCR product was transformed into NTHi 3655 as described (Poje et al., 2003) followed by selection on plates containing chloramphenicol.

### Flow Cytometry Analyses

Bacteria from overnight cultures were grown in broth until OD₆₀₀ 0.8. Thereafter, NTHi strains were washed twice with PBS containing 1% BSA followed by incubation with a purified rabbit anti-pF antiserum according to a standard protocol [Samuelsson et al., 2007]. After washing, bacteria were incubated with FITC-conjugated goat anti-rabbit secondary pAbs (Dakopatts) followed by flow cytometry analysis (EPICS®XL-MCL, Coulter, Hialeah, Fla.).

### Epithelial Cells, Adhesion Assay and Peptide Binding Experiments

Epithelial cell lines were grown to confluence in 24-well plates (Nunc). Bacteria were cultured in BHI with supplements as described above and pulsed with [³H]-thymidine at 36° C. After 4 h, bacteria were washed once with RPMI supplemented with 10% FCS, 0.2% glucose, 0.02% gelatin (reaction medium). The culture medium was removed from the epithelial cells and bacteria (20 μl) in triplicates at different multiplicities of infection (MOI) were added. Thereafter, reaction medium was supplemented followed by centrifugation for 5 min at 800 rpm. After 90 min at 36° C., 5% CO₂, wells were washed 3 times with PBS followed by addition of Trypsin-Versene (Sigma). Triplicate wells were pooled, washed with PBS, transferred to scintillation vials and measured in a beta-counter (Wallac).

A series of synthetic peptides covering the entire pF sequence (FIG. 11) was labeled with [¹²⁵Iodine]-labeling (0.05 mol iodine per mol protein) using the Chloramine T method (Greenwood et al., 1963). The majority of the peptides contained tyrosine residues, but in some cases an extra tyrosine residue was added at the C-terminal. Epithelial cells were incubated with radiolabelled proteins in PBS, 2% BSA for 45 min at 37° C. Thereafter, cells were washed in the same buffer followed by measurement in a γ-scintillation counter.

### Results

Protein F (pF) is a Novel Vitronectin-Binding Protein in Haemophilus influenzae

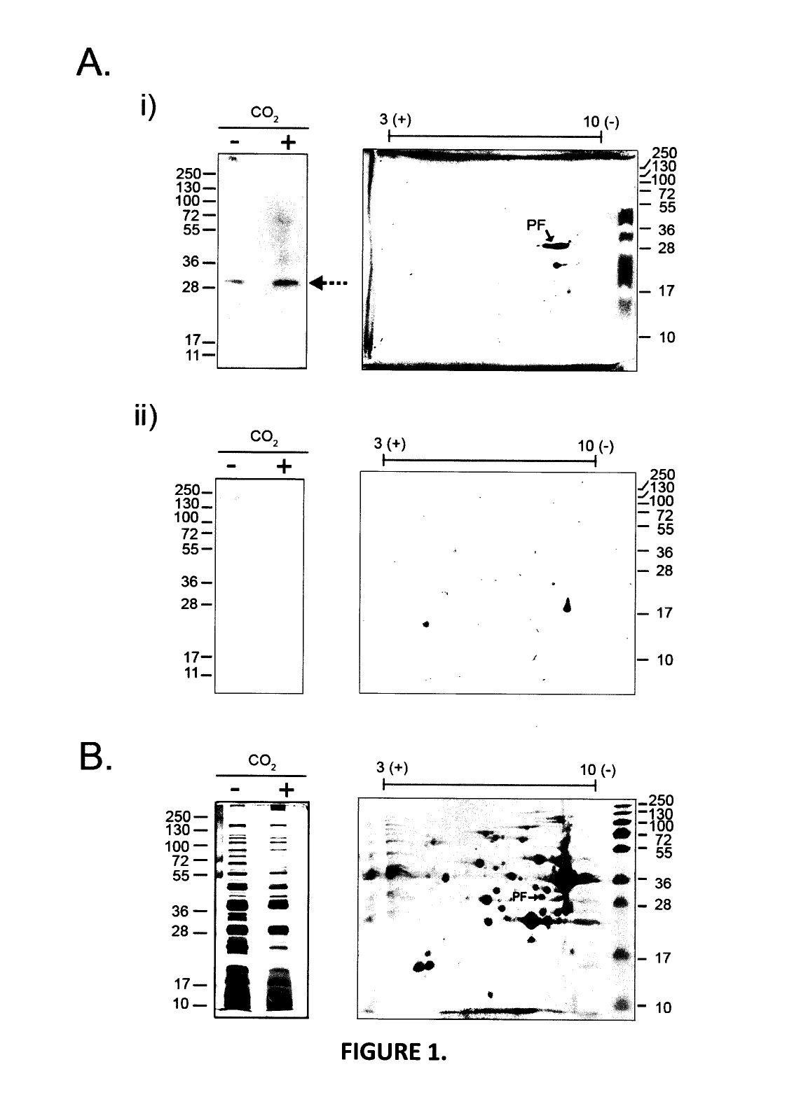

To define vitronectin-binding proteins in non-typable H. influenzae (NTHi), outer membrane vesicles (OMV) were isolated from NTHi 3655. OMV harvested from cultures that were incubated in the absence or presence of CO₂ were subjected to SDS-PAGE. Two gels were run in parallel and one was blotted to a nylon filter (FIG. 1A(i), left panel), whereas the other one was stained with Coomassie blue (FIG. 1B, left panel). The filter was incubated with vitronectin followed by detection using an anti-vitronectin pAb and secondary HRP-conjugated pAb. The strongest signal was obtained with OMV from bacteria incubated with CO₂ (FIG. 1A(i), left panel) that prompted us to run a 2D-SDS-PAGE with the same OMV preparation. As can be seen in FIG. 1A(i) (right panel), a clear signal with vitronectin was obtained on the 2D-SDS-PAGE with OMV from bacteria cultured in the presence of CO₂. To ensure that the detection antibodies did not give a false positive background, filters were also probed with antibodies in the absence of vitronectin (FIG. 1A(ii)). When vitronectin-binding spots had been defined in the 2D-SDS-PAGE (FIG. 1A(i), right panel), corresponding spots were defined on the Coomassie-stained 2D-SDS-PAGE (FIG. 1B, right panel). Several spots were sent for sequencing, and one of the more prominent spots revealed a 29 kDa protein corresponding to H10362 as indicated by the arrows (FIG. 1A(i) and B). This novel vitronectin-binding protein was designated as protein F (pF) and was selected for further analysis.

### the DNA Sequence of Protein F and the Open Reading Frame

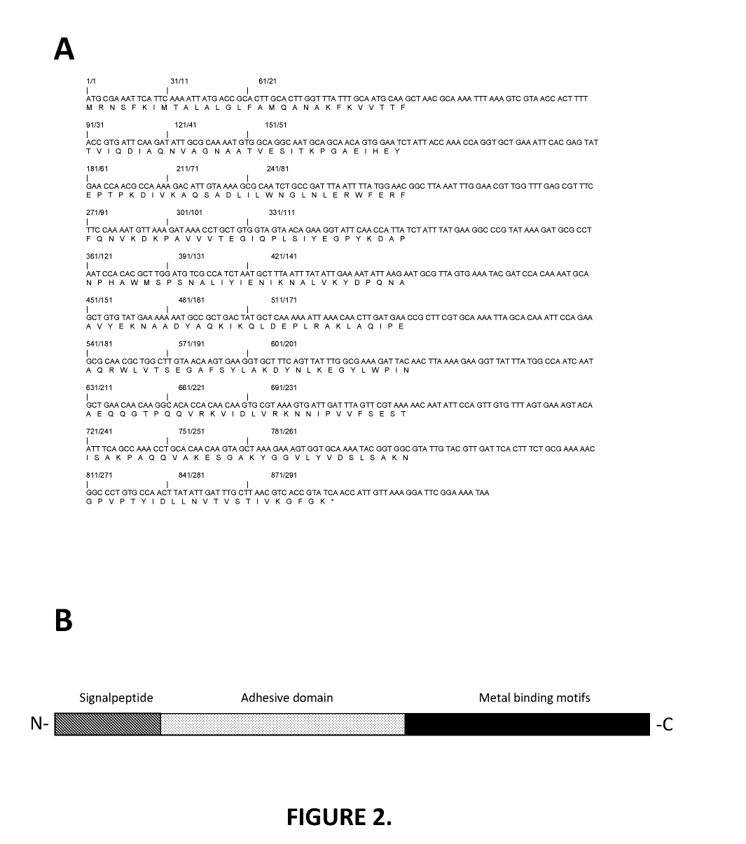

The DNA encoding for protein F in NTHi 3655 was sequenced and analysed in detail. The complete pF sequence consists of 293 amino acids (FIG. 2A) and has a signal peptide that is 22 amino acids in length (FIG. 2B) (Nielsen et al., 1997). The signal peptide is followed by a predicted adhesive domain and a metal binding region at the C-terminal end.

To compare the homology with other pF sequences available in GeneBank, a clustal analysis was done (FIG. 3). Among 15 different pF sequences found, 11 strains had 100% conserved sequences that also included the public sequence of NTHi 3655. Four strains had 99% identity with pF in NTHi 3655. Thus, pF was extraordinary conserved as judged by comparison with other published sequences.

Cloning of Protein F and Expression in E. coli

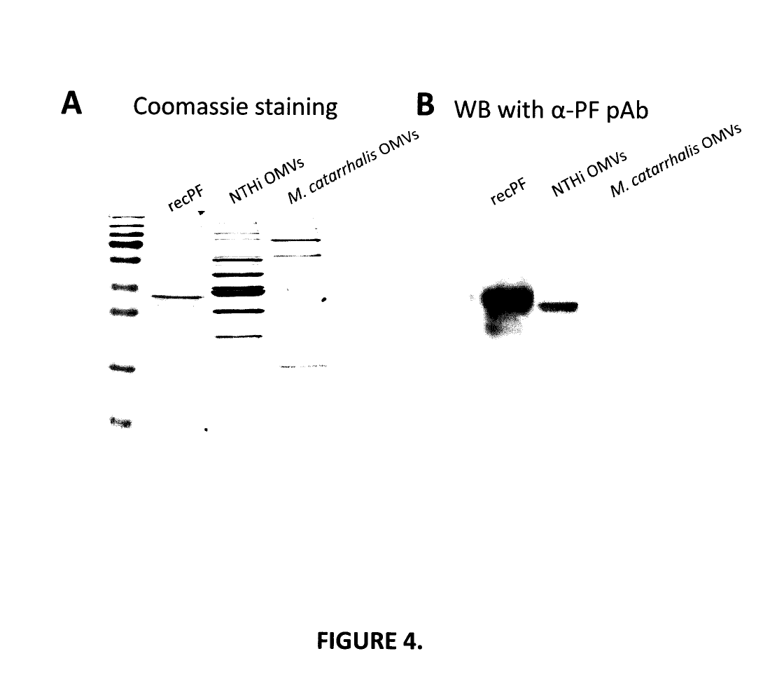

To express pF, the NTHi 3655 genomic sequence was used as template. DNA encoding for the open reading frame (ORF) devoid of the far N-terminal hydrophobic part (11 amino acids) of the signal peptide (pF 1-22) was amplified and cloned into the expression vector pET26 followed by transformation. The resulting recombinant protein was designated pF 12-293. After induction, pF 12-293 was purified by affinity chromatography. Pure protein was eluted and subjected to SDS-PAGE. The resulting recombinant pF with a C-terminal tag consisting of 6 Histidines is demonstrated in FIG. 4A (the 2nd lane from the left). The final product migrated at a size corresponding to approximately 33 kDa.

### Recombinantly Produced Protein F (pF 12-293) is Detected by a Rabbit Antiserum and pE Exists in all Clinical Isolates Analysed

In order to investigate whether pF is immunogenic as well as induce a polyclonal antibody response in rabbits, animals were immunized with recombinant pF12-293. After three immunizations over a total time of 6 weeks, a specific anti-pF antiserum was collected. The presence of PF in the outer membrane of NTHi 3655 was investigated. Outer membrane vesicles (OMVs) were isolated from culture supernatants and subjected to SDS-PAGE. Recombinant pF12-293 as well as OMVs from Moraxella catarrhalis was included on the gel (FIG. 4A). As can be seen in FIG. 4B, the rabbit antiserum recognized both recombinant pF12-293 and native pF in the OMV preparation from NTHi 3655, whereas no cross-reactivity was found with Moraxella OMVs that were included as a negative control. In addition, mice were immunized with pF and these animals also produced an antibody response, that is, specific polyclonal antibodies directed against recombinant pF were detected in ELISA (data not shown).

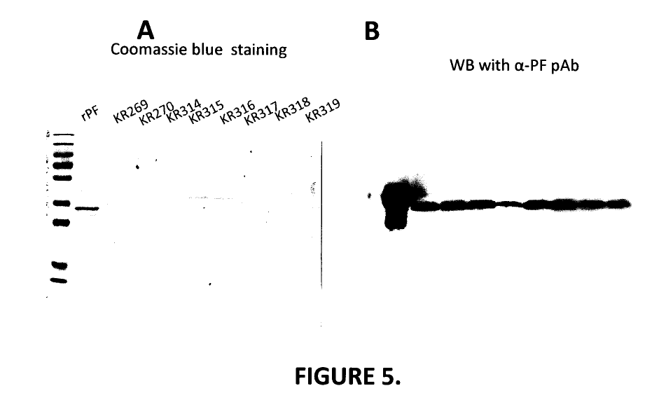

From a vaccine point of view it is important that all clinical NTHi isolates express pF at the protein level. To investigate this, outer membrane proteins were isolated from a series of nasopharyngeal NTHi isolates and subjected to SDS-PAGE followed by blotting (FIG. 5). A Western blot was performed using the specific anti-pF antiserum. Protein F was readily detected with the antiserum and pF was constitutively expressed in all clinical isolates during the standard growth conditions used in the laboratory. The Western blot proved that pF migrated as a single band with the same size (approximately 29 kDa) in all strains analysed (FIG. 5B). To summarize, pF is immunogenic and is found in all clinical NTHi isolates.

### Haemophilus Protein F is a Surface-Exposed Outer Membrane Protein

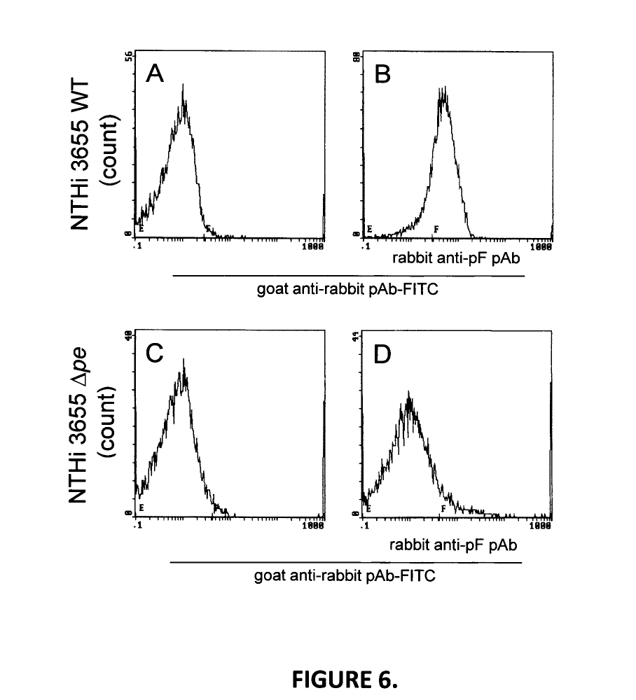

To reveal whether pF was located at the surface of NTHi 3655, a pf-deficient mutant was manufactured by introduction of a gene cassette encoding chloramphenicol acetyltransferase (CAT) resulting in resistance against chloramphenicol. The cat gene cassette was fused with the 5″- and 3″-flanking regions of pF by PCR and overlapping extension. The absence of pF expression was verified by Western blots using the anti-pF antiserum that was purified against recombinant pF 12-293 by chromatography (not shown). Protein F expression was further analysed by flow cytometry (FIG. 6). As can be seen in FIG. 6B, pF was strongly expressed on NTHi 3655 when analysed with the anti-pF pAb as compared to the background control consisting of the FITC-conjugated secondary detection antibody only (FIG. 6A). Interestingly, when the pf gene was mutated, surface exposed pF clearly disappeared (FIG. 6D) as compared to the NTHi 3655 wild type (FIG. 6B). The background control for the pF-deficient NTHi 3655 Δpf mutant is shown in FIG. 6C. These experiments thus proved that pF was located at the bacterial cell surface of H. influenzae.

Protein F is an Outer Membrane Protein that Binds Both Vitronectin and Laminin

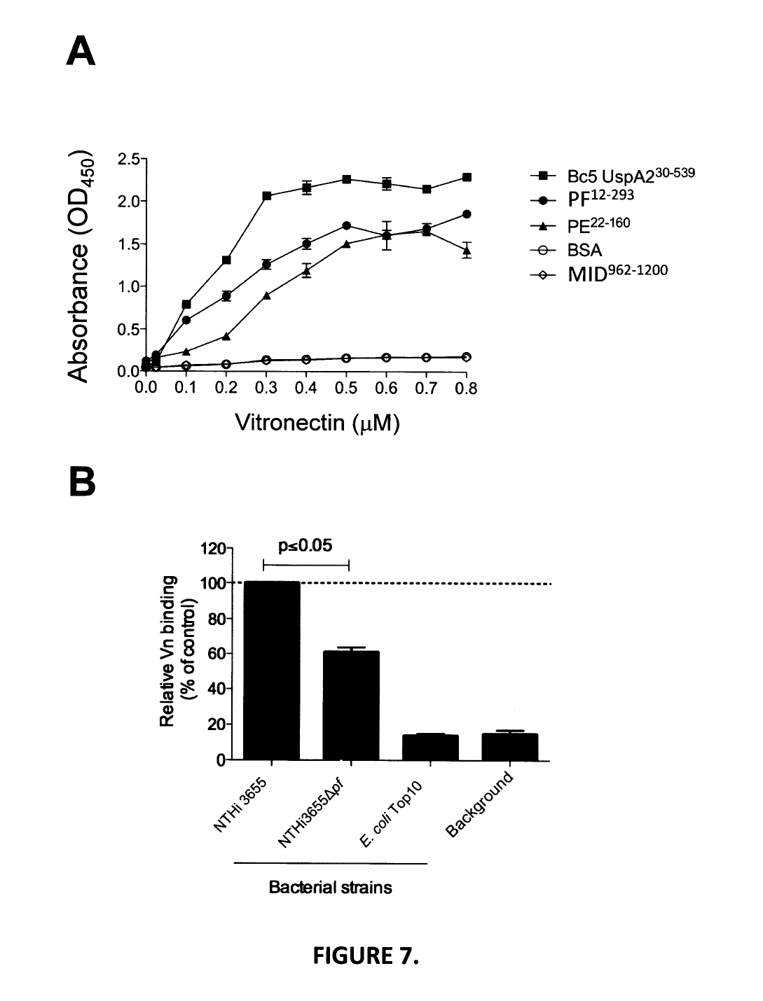

Vitronectin is an important component of the extracellularmatrix (ECM) and also plays a role in maintaining the homeostasis of the complement cascade by inhibiting the formation of the membrane attack complex (MAC) due to mainly binding of complement protein (C) 9 and hence neutralization of the MAC (Singh et al., 2010b). To test whether pF has the capacity to bind vitronectin, recombinant pF12-293 was coated on microtiter plates and tested for binding to full length vitronectin in ELISA. The well-characterized UspA2 (Singh et al., 2010a) and pE (Hallström et al., 2009) were included as positive controls and M. catarrhalis MID962-1200 as a non-vitronectin-binding negative control (Nordström et al, 2002). Interestingly, pF attracted vitronectin slightly better as compared to pE, whereas UspA2 was a stronger binder (FIG. 7A). In contrast, MID 962-1200 did not attract vitronectin in the ELISA.

To further investigate the role of pF-dependent vitronectin binding, the NTHi 3655 Δpf mutant devoid of pF was incubated with [¹²⁵I]-vitronectin in a direct binding assay. Interestingly, the pF-deficient mutant NTHi 3655 Δpf showed a 40% decreased binding to vitronectin when compared to the wild type (FIG. 7B).

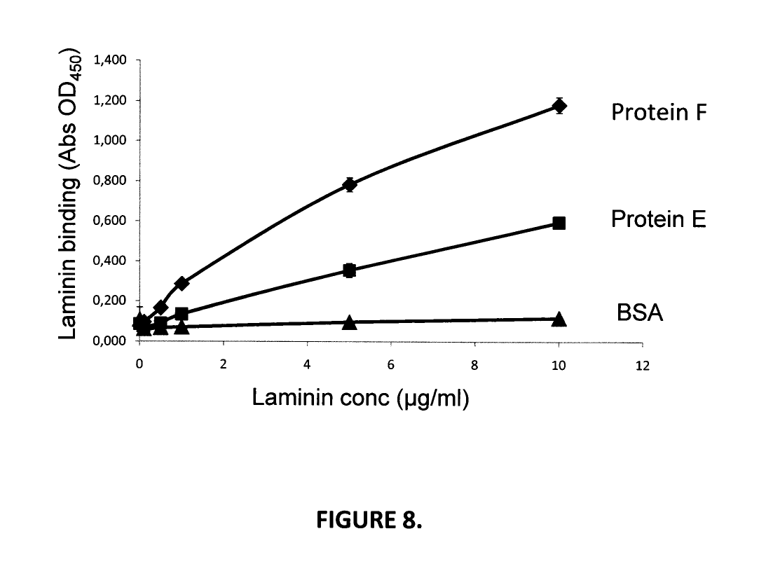

Many bacterial species use the ECM protein laminin as a target molecule on epithelial cells. To test whether recombinant protein F also binds laminin, recombinant pF 12-293 was coated in microtiter plates followed by addition of laminin and specific detection with an anti-laminin pAb. A dose response was seen and pF bound laminin slightly better as compared to protein E (FIG. 8). Taken together, Haemophilus pF binds to both vitronectin and laminin and thus is an important virulence factor in NTHi pathogenesis.

Protein F Attaches to Epithelial Cells and the Active Binding Domain is Located within the N-Terminal Part of Protein F (Amino Acids pF23-48)

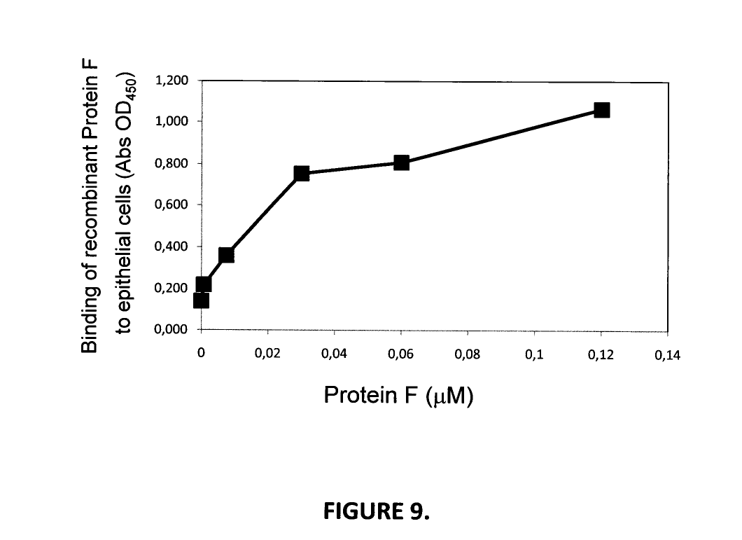

Several vitronectin-binding proteins also play a role in attachment to epithelial cells (Singh et al., 2010b), that is, these often multifunctional outer membrane proteins work as adhesins. To test whether pF also can promote bacterial binding to epithelial cells, recombinant pF12-293 was added to epithelial cells attached to a plastic surface. A dose-response was observed when increasing concentrations up to 0.12 μM of pF12-293 was added to cells followed by detection using the anti-pF rabbit antiserum and secondary HRP-conjugated detection antibodies (FIG. 9).

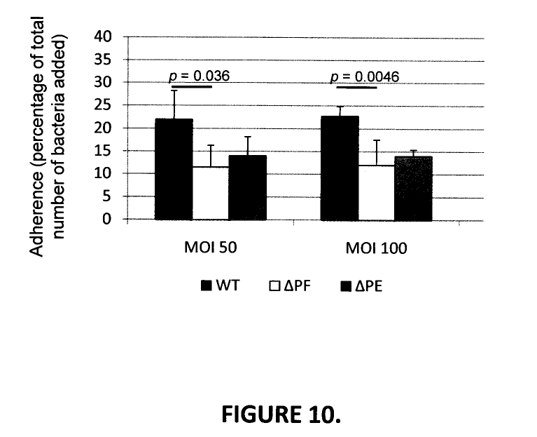

To further prove the importance of pF as an adhesin, the pF-deficient mutant NTHi 3655 Δpf was compared to the pF-expressing native NTHi 3655 wild type. Interestingly, the pF-mutant lost more than 50% of its binding capacity when analysed with epithelial cells at a multiplicity of infection (MOI) at 50 and 100 (FIG. 10) further proving that pF is an important NTHi adhesin. Thus, pF played a role in attachment of NTHi to epithelial cells and can be considered as a bacterial adhesin.

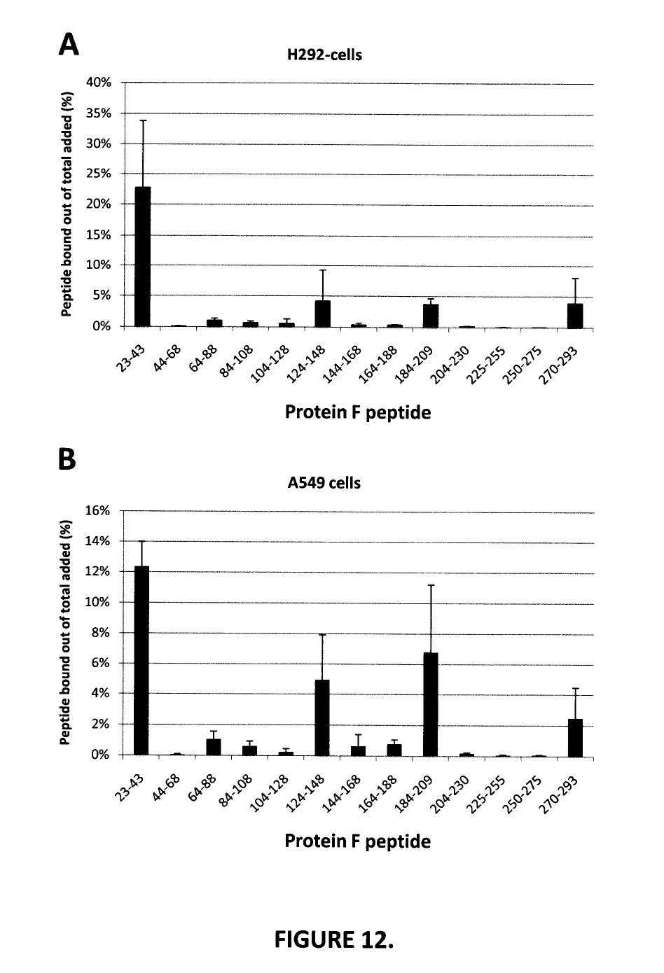

A series of synthetic peptides was manufactured to analyse the precise binding region of pF that was responsible for attachment to the epithelial cell surface (FIG. 11). The peptides were labelled with iodine and incubated with two different epithelial cell lines. As can be seen in FIG. 12, the N-terminal peptide (pE 23-48) significantly bound to both H292 (FIG. 12A) and A549 epithelial cells (FIG. 12B). In conclusion, the main epithelial binding site was located within the N-terminal part of the molecule.

The N-Terminal pF44-68 is Surface Exposed and can be Detected by Specific Antibodies.

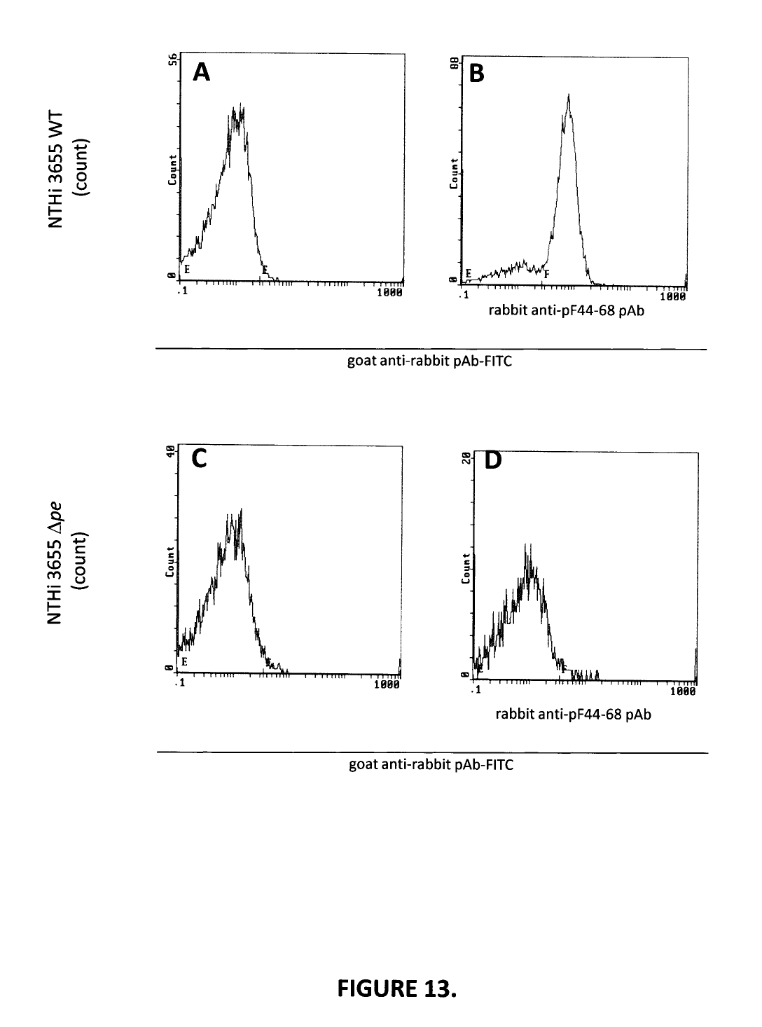

Since the epithelial cell binding site most likely is located in the N-terminal part of pF this part of the molecule would be surface exposed. Bioinformatics revealed that pF23-48 was not immunogenic (not shown). In contrast, pF44-68 would be immunogenic as judged by the analysis. Therefore, the peptide pF44-68 was coupled to CnBr-Sepharose followed by absorption of specific anti-pF44-68 pAb using the pF12-293 antiserum as a source. Both the NTHi3655 wild type and the pF-deficient mutant were incubated with the resulting anti-pF44-68 rabbit pAb. Thereafter, FITC-conjugated goat anti-rabbit detection antibodies were added followed by flow cytometry analysis. As can be seen in FIG. 13B, pF-expressing NTHi 3655 was readily detected with the anti-pF44-68 pAb. In contrast, the pF-deficient mutant NTHi 3655 Δpf mutant was not detected with the anti-pF44-68 pAb (FIG. 13D). Bacteria incubated in the absence of the anti-pF44-68 pAb are also shown and proved that the secondary pAb did not bind (FIGS. 13A and C). Taken together, the N-terminal part of pF can be found at the surface by recognition by antibodies directed against the sequence pF44-68.

### REFERENCES

Greenwood F C, Hunter W M and Glover J S. The Preparation of I-131-Labelled Human Growth Hormone of High Specific Radioactivity. Biochem J 1963; 89:114-23.
Hallström T, Blom A M, Zipfel P F, Riesbeck K. Nontypeable Haemophilus influenzae protein E binds vitronectin and is important for serum resistance. J Immunol. 2009 Aug. 15; 183(4):2593-601. Epub 2009 Jul. 27. PubMed PMID: 19635912.
Heino J, Kapyla J. Cellular receptors of extracellular matrix molecules. Curr Pharm Des 2009; 15:1309-17.
Nielsen, H., J. Engelbrecht, S. Brunak, and G. von Heijne. 1997. Identification of prokaryotic and eukaryotic signal peptides and prediction of their cleavage sites. Protein Engineering 10: 1.
Nordstrom T, Forsgren A and Riesbeck K. The immunoglobulin D-binding part of the outer membrane protein MID from Moraxella catarrhalis comprises 238 amino acids and a tetrameric structure. J Biol Chem 2002; 277:34692-9.
Janson, H., L.-O. Hedén, A. Grubb, M. Ruan, and A. Forsgren. 1991. Protein D, an immunoglobulin D-binding protein of Haemophilius influenzae: cloning, nucleotide sequence, and expression in Escherichia coli. Infect. Immun. 59:119.
Kinhikar A G, Vargas D, Li H, et al. Mycobacterium tuberculosis malate synthase is a laminin-binding adhesin. Mol Microbiol 2006; 60:999-1013.
Nguyen N M, Senior R M. Laminin isoforms and lung development: all isoforms are not equal. Dev Biol 2006; 294:271-9.
Poje G, J. R. Redfield, Transformation of Haemophilus influenzae, Methods. Mol. Med. 71 (2003) 57-70.
Preissner, K. T., E. R. Podack, and H. J. Muller-Eberhard. 1985. The membrane attack complex of complement: relation of C7 to the metastable membrane binding site of the intermediate complex C5b-7. J. Immunol. 135:445-451.
Preissner, K. T. 1991. Structure and biological role of vitronectin. Annu. Rev. Cell. Biol. 7:275-310.
Preissner, K. T. and Seiffert, D. (1998). Role of vitronectin and its receptors in haemostasis and vascular remodeling. Thromb Res 89, 1-21.

Prymula R, Peeters P, Chrobok V, Kriz P, Novakova E, Kaliskova E, Kohl I, Lommel P, Poolman J, Prieels J P, Schuerman L. (2006). Pneumococcal capsular polysaccharides conjugated to protein D for prevention of acute otitis media caused by both Streptococcus pneumoniae and non-typable Haemophilus influenzae: a randomised double-blind efficacy study. Lancet 367, 740-8.
Riesbeck K, Chen D, Kemball-Cook G, McVey J H, George A J, Tuddenham E G, Dorling A, Lechler R I. Expression of hirudin fusion proteins in mammalian cells: a strategy for prevention of intravascular thrombosis. Circulation. 1998; 98(24):2744-52. PubMed PMID: 9851962.
Ronander E, Brant M, Janson H, Sheldon J, Forsgren A and Riesbeck K. Identification of a novel Haemophilus influenzae protein important for adhesion to epithelial cells. Microbes Infect 2008; 10:87-96.
Ronander E, Brant M, Eriksson E, Mörgelin M, Hallgren O, Westergren-Thorsson G, Forsgren A, Riesbeck K. Nontypeable Haemophilus influenzae adhesin protein E: characterization and biological activity. J Infect Dis. 2009 Feb. 15; 199(4):522-31. PubMed PMID: 19125675.
Samuelsson M, Hallström T, Forsgren A, Riesbeck K. Characterization of the IgD binding site of encapsulated Haemophilus influenzae serotype b. J Immunol. 2007; 178(10):6316-9. PubMed PMID: 17475860.
Schaar V, De Vries S P, Perez Vidakovics M L, Bootsma H J, Larsson L, Hermans P W, Bjartell A, Mörgelin M, Riesbeck K. Multicomponent Moraxella catarrhalis outer membrane vesicles induce an inflammatory response and are internalized by human epithelial cells. Cell Microbiol. 2010 Nov. 2. doi: 10.1111/j.1462-5822.2010.01546.x. PubMed PMID: 21044239.
Singh B, Blom A M, Unal C, Nilson B, Mörgelin M, Riesbeck K. Vitronectin binds to the head region of Moraxella catarrhalis ubiquitous surface protein A2 and confers complement-inhibitory activity. Mol Microbiol. 2010 March; 75(6):1426-44. Epub 2010 Feb. 19. PubMed PMID: 20199596.
Singh B, Su Y C, Riesbeck K. Vitronectin in bacterial pathogenesis: a host protein used in complement escape and cellular invasion. Mol Microbiol. 2010 November; 78(3):545-60. doi: 10.1111/j.1365-2958.2010.07373.x. Epub 2010 Sep. 27. PubMed PMID: 20807208.
Smith, H. W. and Marshall, C. J. Regulation of cell signalling by uPAR. (2010). Nat Rev Mol Cell Biol 11, 23-36.
Tan T T, Forsgren A and Riesbeck K. The respiratory pathogen Moraxella catarrhalis binds to laminin via ubiquitous surface proteins A1 and A2. J Infect Dis 2006; 194:493-7.
Vidakovics M L, Jendholm J, Mörgelin M, Månsson A, Larsson C, Cardell L O, Riesbeck K. B cell activation by outer membrane vesicles—a novel virulence mechanism. PLoS Pathog. 2010 Jan. 15; 6(1):e1000724. PubMed PMID: 20090836; PubMed Central PMCID: PMC2799554.

1. An immunogenic composition comprising a polypeptide, or a fragment thereof selected from the group consisting of:
a) a polypeptide comprising an amino acid sequence which has at least 85% identity to the amino acid sequence set forth in SEQ ID NO: 1, or a fragment thereof comprising at least 15 contiguous amino acids;
b) a polypeptide comprising the amino acid sequence set forth in any one of SEQ ID NOS: 1, 2, 3, 4, 5, 6, 7, 8, 9, 10, 11, 12, 13, or 14, or a fragment thereof comprising at least 15 contiguous amino acids; and
c) a polypeptide consisting of the amino acid sequence set forth in any one of SEQ ID NOS: 1, 2, 3, 4, 5, 6, 7, 8, 9, 10, 11, 12, 13, or 14, or a fragment thereof comprising at least 15 contiguous amino acids; and
a pharmaceutically acceptable excipient.

2. (canceled)

3. The immunogenic composition of claim 1, wherein one or more of the amino acids in position 1-11 or 1-22 of SEQ ID NO: 1 have been deleted or replaced by one or more amino acids.

4-17. (canceled)

18. The immunogenic composition-of claim 1, wherein the polypeptide, or fragment thereof, is present as a dimer, trimer, or multimer.

19. The immunogenic composition-of claim 1, further comprising one or more pharmaceutically acceptable adjuvants, vehicles, excipients, binders, carriers, preservatives, buffering agents, emulsifying agents, wetting agents, or transfection facilitating compounds.

20-22. (canceled)

23. An immunogenic composition comprising a nucleic acid molecule comprising a nucleotide sequence selected from the group consisting of:
a) a nucleotide sequence comprising at least 85% identity to the nucleotide sequence set forth in SEQ ID NO: 15;
b) a nucleotide sequence comprising the nucleotide sequence set forth in SEQ ID NO: 15;
c) a nucleotide sequence consisting of the nucleotide sequence set forth in SEQ ID NO: 15;
d) a nucleotide sequence encoding a polypeptide comprising an amino acid sequence which has at least 85% identity to the amino acid sequence set forth in SEQ ID NO:1, or a fragment thereof comprising-at least 15 contiguous amino acids;
e) a nucleotide sequence encoding a polypeptide comprising the amino acid sequence set forth in any one of SEQ ID NOS: 1, 2, 3, 4, 5, 6, 7, 8, 9, 10, 11, 12, 13, or 14, or a fragment thereof comprising at least 15 contiguous amino acids;
f) a nucleotide sequence encoding a polypeptide consisting of the amino acid sequence set forth in any one of SEQ ID NOS:1, 2, 3, 4, 5, 6, 7, 8, 9, 10, 11, 12, 13, or 14, or a fragment thereof comprising at least 15 contiguous amino acids; and
g) a nucleotide sequence complementary to any of the nucleotide sequences of subpart (a)-(f), and
a pharmaceutically acceptable excipient.

24. The immunogenic composition of claim 23, wherein the nucleic acid, is fused to at least another gene.

25. The immunogenic composition of claim 23, wherein the nucleic acid is in a plasmid or phage.

26-49. (canceled)

50. A process for producing an immunogenic composition of comprising culturing a host cell comprising a nucleic acid molecule comprising a nucleotide sequence selected from the group consisting of:
a) a nucleotide sequence comprising at least 85% identity to the nucleotide sequence set forth in SEQ ID NO: 15;
b) a nucleotide sequence comprising the nucleotide sequence set forth in SEQ ID NO: 15;
c) a nucleotide sequence consisting of the nucleotide sequence set forth in SEQ ID NO: 15;
d) a nucleotide sequence encoding a polypeptide comprising an amino acid sequence which has at least 85% identity to the amino acid sequence set forth in SEQ ID NO:1, or a fragment thereof, comprising-at least 15 contiguous amino acids;
e) a nucleotide sequence encoding a polypeptide comprising the amino acid sequence set forth in any one of SEQ ID NOS: 1, 2, 3, 4, 5, 6, 7, 8, 9, 10, 11, 12, 13, or 14, or a fragment thereof, comprising at least 15 contiguous amino acids;
f) a nucleotide sequence encoding a polypeptide consisting of the amino acid sequence set forth in any one of SEQ ID NOS:1, 2, 3, 4, 5, 6, 7, 8, 9, 10, 11, 12, 13, or 14, or a fragment thereof, comprising at least 15 contiguous amino acids; and
g) a nucleotide sequence complementary to any of the nucleotide sequences of subparts (a)-(e)
under conditions sufficient for production of a polypeptide from the nucleotide sequence; recovering the polypeptide from the culture medium; and
mixing the polypeptide with a pharmaceutically acceptable excipient.

51-53. (canceled)

54. The immunogenic composition of claim 1, wherein said composition comprises at least one other antigen.

55. The immunogenic composition of claim 54, where the other antigen is from Haemophilus influenza, Streptococcus pneumonia, or Moraxella catarrhalis.

56-70. (canceled)

71. A method of isolating a polypeptide comprising the amino acid sequence set forth in any one of SEQ ID NOS: 1-14, or a fragment thereof, said method comprising the steps of:
a) growing Haemophilus influenzae or E. coli comprising the DNA coding for said polypeptide, or fragment thereof, harvesting the Haemophilus influenzae or E. coli and isolating outer membranes or inclusion bodies;
b) solubilizing the inclusion bodies with a strong solvatising agent;
c) adding a renaturating agent;
d) dialyzing the resulting suspension against a buffer with a pH from 8 to 10; and
e) isolating the polypeptide.

72-74. (canceled)

75. A method of preventing or treating an infection in an individual comprising administering a pharmaceutically effective amount of the immunogenic composition according to claim 1.

76. The method according to claim 75, wherein the infection is caused by Haemophilus influenzae.

77. The method according to claim 76, wherein the Haemophilus influenzae is encapsulated or non-typable.

78. The method according to claim 75, wherein the infection is otitis media, sinusitis, or a lower respiratory tract infection.

79. The method according to claim 75, wherein the individual suffers from chronic obstructive pulmonary disease.


SEQUENCE LISTING (15 sequences)

SEQ ID NO: 1 - protein, 293 aa, Haemophilus influenzae (pF)
MRNSFKIMTALALGLFAMQANAKFKVVTTFTVIQDIAQNVAGNAATVESITKPGAEIHEYEPTPKDIVKAQSADLILWNGLNLERWFERFFQNVKDKPAVVVTEGIQPLSIYEGPYKDAPNPHAWMSPSNALIYIENIKNALVKYDPQNAAVYEKNAADYAQKIKQLDEPLRAKLAQIPEAQRWLVTSEGAFSYLAKDYNLKEGYLWPINAEQQGTPQQVRKVIDLVRKNNIPVVFSESTISAKPAQQVAKESGAKYGGVLYVDSLSAKNGPVPTYIDLLNVTVSTIVKGFGK

SEQ ID NO: 2 - protein, 26 aa, Artificial Sequence
  Protein fragment from Haemophilus Influenzae (pF) or synthetic protein.
KFKVVTTFTVIQDIAQNVAGNAATVE

SEQ ID NO: 3 - protein, 25 aa, Artificial Sequence
  Protein fragment from Haemophilus Influenzae (pF) or synthetic protein.
AATVESITKPGAEIHEYEPTPKDIV

SEQ ID NO: 4 - protein, 25 aa, Artificial Sequence
  Protein fragment from Haemophilus Influenzae (pF) or synthetic protein.
PKDIVKAQSADLILWNGLNLERWFE

SEQ ID NO: 5 - protein, 25 aa, Artificial Sequence
  Protein fragment from Haemophilus Influenzae (pF) or synthetic protein.
ERWFERFFQNVKDKPAVVVTEGIQP

SEQ ID NO: 6 - protein, 25 aa, Artificial Sequence
  Protein fragment from Haemophilus Influenzae (pF) or synthetic protein.
EGIQPLSIYEGPYKDAPNPHAWMSP

SEQ ID NO: 7 - protein, 25 aa, Artificial Sequence
  Protein fragment from Haemophilus Influenzae (pF) or synthetic protein.
AWMSPSNALIYIENIKNALVKYDPQ

SEQ ID NO: 8 - protein, 25 aa, Artificial Sequence
  Protein fragment from Haemophilus Influenzae (pF) or synthetic protein.
KYDPQNAAVYEKNAADYAQKIKQLD

SEQ ID NO: 9 - protein, 25 aa, Artificial Sequence
  Protein fragment from Haemophilus Influenzae (pF) or synthetic protein.
IKQLDEPLRAKLAQIPEAQRWLVTS

SEQ ID NO: 10 - protein, 26 aa, Artificial Sequence
  Protein fragment from Haemophilus Influenzae (pF) or synthetic protein.
WLVTSEGAFSYLAKDYNLKEGYLWPI

SEQ ID NO: 11 - protein, 28 aa, Artificial Sequence
  Protein fragment from Haemophilus Influenzae (pF) or synthetic protein.
GYLWPINAEQQGTPQQVRKVIDLVRKNN

SEQ ID NO: 12 - protein, 32 aa, Artificial Sequence
  Protein fragment from Haemophilus Influenzae (pF) or synthetic protein.
YDLVRKNNIPVVFSESTISAKPAQQVAKESGA

SEQ ID NO: 13 - protein, 26 aa, Artificial Sequence
  Protein fragment from Haemophilus Influenzae (pF) or synthetic protein.
AKESGAKYGGVLYVDSLSAKNGPVPT

SEQ ID NO: 14 - protein, 24 aa, Artificial Sequence
  Protein fragment from Haemophilus Influenzae (pF) or synthetic protein.
NGPVPTYIDLLNVTVSTIVKGFGK

SEQ ID NO: 15 - DNA, 882 bp, Haemophilus influenzae (pF)
atgcgaaattcattcaaaattatgaccgcacttgcacttggtttatttgcaatgcaagctaacgcaaaatttaaagtcgtaaccacttttaccgtgattcaagatattgcgcaaaatgtggcaggcaatgcagcaacagtggaatctattaccaaaccaggtgctgaaattcacgagtatgaaccaacgccaaaagacattgtaaaagcgcaatctgccgatttaattttatggaacggcttaaatttggaacgttggtttgagcgtttcttccaaaatgttaaagataaacctgctgtggtagtaacagaaggtattcaaccattatctatttatgaaggcccgtataaagatgcgcctaatccacacgcttggatgtcgccatctaatgctttaatttatattgaaaatattaagaatgcgttagtgaaatacgatccacaaaatgcagctgtgtatgaaaaaaatgccgctgactatgctcaaaaaattaaacaacttgatgaaccgcttcgtgcaaaattagcacaaattccagaagcgcaacgctggcttgtaacaagtgaaggtgctttcagttatttggcgaaagattacaacttaaaagaaggttatttatggccaatcaatgctgaacaacaaggcacaccacaacaagtgcgtaaagtgattgatttagttcgtaaaaacaatattccagttgtgtttagtgaaagtacaatttcagccaaacctgcacaacaagtagctaaagaaagtggtgcaaaatacggtggcgtattgtacgttgattcactttctgcgaaaaacggccctgtgccaacttatattgatttgcttaacgtcaccgtatcaaccattgttaaaggattcggaaaataa